(19) United States
(12) Patent Application Publication
(10) Pub. No.: US 2007/0142462 A1
(43) Pub. Date: Jun. 21, 2007

(54) Method of Treating Cancer

(75) Inventor: Thomas Preston Kennedy, Charlotte, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of Utah

(21) Appl. No.: 11/671,199

(22) Filed: Feb. 5, 2007

Related U.S. Application Data
(62) Division of application No. 10/258,308, filed on Sep. 3, 2003.
(60) Provisional application No. 60/200,028, filed on Apr. 26, 2000.

(51) Int. Cl. A61K 31/366; A61K 31/12

(52) U.S. Cl. 514/457; 514/682

(57) ABSTRACT

Methods for treating cancer are provided including administering a patient needing treatment a therapeutically effective amount of one or more antioxidants selected from the group of catalase, N-acetylcysteine, glutathione peroxidase, salen-transition metal complexes, dicumarol, and derivatives thereof.

### CROSS REFERENCE TO RELATED APPLICATIONS

The present application is a divisional of application Ser. No. 10/258,308, filed on Sep. 3, 2003, which is a 35 U.S.C §371 national stage application of PCT Appl. No. PCT/US01/40237, filed on Mar. 2, 2001, which claims the benefit of U.S. application Ser. No. 60/200,028, filed on Apr. 26, 2000, all of which are incorporated by reference in their entirety and for all purposes.

### FIELD OF INVENTION

This invention generally relates to methods of treating cancer, and particularly to methods of treating cancer by inhibiting NF-κB activities with compounds including antioxidants.

### BACKGROUND OF THE INVENTION

Cancer, the uncontrolled growth of malignant cells, is a major health problem of the modern medical era and ranks second only to heart disease as a cause of death in the U.S. While some malignancies, such as adenocarcinoma of the breast and lymphomas such as Hodgkins Disease, respond relatively well to current chemotherapeutic antineoplastic drug regimens, other cancers are poorly responsive to chemotherapy, especially non-small cell lung cancer and pancreatic, prostate and colon cancers. Even small cell cancer of the lung, initially chemotherapy sensitive, tends to return after remission, with widespread metastatic spread leading to death of the patient. Thus, better treatment approaches are needed for this illness. Also, because almost all currently available antineoplastic agents have significant toxicities, such as bone marrow suppression, renal dysfunction, stomatitis, enteritis and hair loss, it would be of major advantage to have a relatively less toxic agent available for use alone or in combination with current drugs in order to better treat the patient without risking injury from the therapy itself.

There is a growing body of evidence indicating important functions of nuclear factor-κB (NF-κB) in growth control and cell cycle regulation especially in tumor cells. Constitutive nuclear NF-κB activation has recently been reported as important for proliferation of a number of malignancies, including carcinomas of breast, See Sovak et al., J. Clin. Invest. 100:2952-2960 (1997); ovary, See Bours et al., Biochem. Pharmacol. 47:145-149 (1994); colon, See Bours et al., Biochem. Pharmacol. 47:145-149 (1994); head and neck, See Duffey et al., Cancer Res. 59:3468-3474 (1994); pancrease, See Wang et al., Clin. Cancer Res. 5:110-127 (1999); melanoma, See Shattuck-Brandt et al., Cancer Res. 57:3032-3039 (1997); and Hodgkin's disease, See Bargou et al., J. Clin. Invest. 100:2961-2969 (1997). This is in contrast to the transient nuclear NF-κB activity observed in most normal cell types which is caused by certain inducers. Activation of NF-κB has also been linked with resistance of tumors to TNF-α-induced apoptosis, anti-cancer chemotherapy and radiation, See Wang et al., Science, 274:784-789 (1996); Beg et al., Science 274:782-784 1996); and Wang et al., Nature Med. 5:412-417 (1999).

NF-κB is commonly known to be an important transcription factor involved in the regulation of a variety of genes in animal cells. Normally in a quiescent state, NF-κB resides in the cytoplasm in the form of a “Rel complex”. When activated by an extracellular or intracellular signal, NF-κB translocates to the nucleus, where it attaches to cis-acting κB sites in promoters and enhancers of a variety of genes. The NF-κB binding DNA consensus sequence is 5′-GGGPuNNPyPyCC-3′. The translocation of NF-κB upregulates the transcription of mRNA for a host of proteins. An important group of such proteins are cytokines including tumor necrosis factor (TNF), IL-1, IL-2, IL-6, IL-8, interferon-β, interferon-γ, tissue factor-1, complement, and inducible nitric oxide synthase, and the like. See e.g., Siebenlist et al., Annu. Rev. Cell Biol. 10:405-455 (1994); see also U.S. Pat. No. 5,804,374.

In cytoplasm, NF-κB is associated with the inhibitory molecules I-κBs in the Rel complex. See, e.g., Grimm, et al., Biochem. J. 290:297-308 (1993). Genes encoding both NF-κB and a number of I-κBs have been isolated. See, e.g., U.S. Pat. Nos. 5,804,374; 5,597,898; 5,849,580. Activation of NF-κB is initiated when I-κB is phosphorylated by I-κB kinase. The phosphorylation leads to the recognition of I-κB by ubiquitin and subsequent proteosomal degradation. See, e.g., Thanos et al. Cell 80:529-532 (1995); Stancovski, et al., Cell, 91:299-302 (1997). The removal of I-κB from the NF-κB protein exposes a positively charged group of amino acids on NF-κB protein known as the nuclear localization site (NLF), thus allowing the translocation of NF-κB to nucleus.

It has been found that the nuclear translocation of NF-κB can be competitively inhibited in a dose-dependent manner by synthetic peptides containing a cell membrane permeable motif and the nuclear localization sequence. See Lin et al., J. Biol. Chem. 270:14255-14258 (1995). A number of glucocorticoids have been shown to be able to block the translocation of NF-κB from the cytoplasm to nucleus. See Adcock, et al., Am. J. Physiol. 37:C331-C338 (1995); see also Ray et al., Proc. Natl. Acad. Sci. USA 91:752-756 (1994).

However, glucocorticoids when used in patients have a number of adverse effects, including induction of hypertension, glucose intolerance and bone demineralization. Thus, it would be of major advantage to develop an alternative, non-glucocorticoid-based approach for inhibiting activation of NF-κB, which may provide to be a new strategy in treating diseases in animals.

### SUMMARY OF THE INVENTION

The present invention provides a method for treating cancer in a patient by administering to the patient a pharmaceutical composition containing a therapeutically effective amount of an antioxidant capable of inhibiting the activation of cellular NF-κB.

In accordance with a first aspect of the present invention, a therapeutically effective amount of one or more antioxidants selected from the group of catalase, N-acetylcysteine, glutathione peroxidase, salen-transition metal complexes, and derivatives thereof are administered to a patient for the purpose of treating cancer.

In addition, it has been discovered that cellular NAD(P)H:quinone oxidoreductase (NQO, EC 1.6.99.2) is a key enzyme for generating O₂⁻ in tumor cells and may represent the common source of reactive oxygen species that cause the activation of NF-κB and malignant cell growth. Accordingly, in accordance with another aspect of the present invention, a therapeutically effective amount of an inhibitor of NAD(P)H:quinone oxidoreductase is administered to a patient for the purpose of treating cancer in the patient. Preferably, dicumarol, a clinically tested anticoagulant, is administered in accordance with this aspect of the invention.

In a preferred embodiment, a therapeutically effective amount of dicumarol is administered with a member of the vitamin K family. Dicumarol can be administered simultaneously in the same pharmaceutical preparation with vitamin K. Dicumarol and vitamin K can also be administered at about same time but by a separate administration. Alternatively, dicumarol can be administered at a different time from the administration of vitamin K.

While not wishing to be bound by any theories, it is believed that addition of antioxidants including dicumarol, N-acetylcysteine, catalase, glutathione peroxidase, salen-transition metal complexes, and derivatives thereof to tumor cells inhibits the formation of, or catalytically or stoichiometrically removes hydrogen peroxide generated in tumor cells, and inhibits the activation of NF-κB in tumor cells. Thus, they function to decrease the level of reactive oxygen species, in particular hydrogen peroxide, in cytoplasm of the cells and prevent NF-κB from being accumulated in cell nucleus. As a result, cell growth of the malignant cells is substantially inhibited.

The active compounds of this invention can be formulated with a pharmaceutically acceptable carrier and administered through a variety of administration routes. For example, they can be administered orally, intravenously, intradermally, subcutaneously and topically.

The method of the present invention can be used in treating various types of cancer, and will be especially effective in treating melanoma, prostate carcinoma, and breast carcinoma.

Many of the active compounds used in the method of this invention either have been clinically tested to be safe or have been used for other diseases unrelated to cancer. The toxicology and pharmacology profiles of many of these compounds are known. Thus, the discovery of the new use of these compounds in this invention offers a readily available and easily used treatment for cancer in man and other mammals.

The foregoing and other advantages and features of the invention, and the manner in which the same are accomplished, will become more readily apparent upon consideration of the following detailed description of the preferred embodiments of the invention taken in conjunction with the accompanying examples, which illustrate preferred and exemplary embodiments.

### BRIEF DESCRIPTION OF THE DRAWINGS

FIGS. 1A-1D are diagrams showing the antiproliferation effect of catalase, N-acetylcyteine, and ebselen;

FIGS. 2A-2B are photographs of DNase-treated M1619 cells (FIG. 2A) and catalase-treated M1619 cells (FIG. 2B) labeled with a Fluorescein-FragEL™ DNA fragmentation detection kit (Oncogene Research Products), demonstrating that catalase treatment does not induce apoptosis or necrosis;

FIGS. 3A-3E are autoradiograms from electrophoresis mobility shift assays on malignant cell lines either untreated or treated with catalase, ebselen, N-acetylcyteine or dicumarol, indicating that antioxidant treatment reduces constitutive nuclear DNA binding activity for NF-κB in malignant cell lines;

FIGS. 4A-4D are photographs showing immunohistochemical staining pattern of p65 (a component of NF-κB) in M1619 cells untreated (FIG. 4A), or treated with catalase (FIG. 4B), apocynin (FIG. 4C) or dicumarol (FIG. 4D);

FIG. 5A is a gel autoradiogram obtained from immunoassays for phosphorylated IκBα (IκBα-P) using phospho-specific antibody in untreated and catalase-treated M1619 cells;

FIG. 5B is a graph showing the mean ratios of the densitometrically determined intensities of IκBα-P staining in the immunoassay experiments of FIG. 5A;

FIGS. 6A and 6B are diagrams obtained from DNA cell cycle analysis of M1619 cells untreated (FIG. 6A) or treated with catalase (FIG. 6B), using FACSStar^{PLUS} Flow Cytometer;

FIGS. 7A-7D illustrate the effect of catalase in reducing expression of cyclin B1 and p34-cdc2 kinase in M1619 melanoma cells. A. Time course of the effect of catalase on cyclin B1 expression. Lanes 1-6 and 7-12 represent treatment for 2, 4, 8, 12, 24 and 48 hours, of untreated control or catalase-treated cells, respectively. B. Densitometry summary of 4 experiments per group where cells were treated for 24 hours with catalase. *p<0.001 vs untreated control cells. C. Time course of the effect of catalase on p34-cdc2 expression. Lanes 1-9 and 10-18 represent treatment for 15 min, 30 min and 1, 1.5, 2, 4, 8, 12 or 24 hours, of untreated control or catalase-treated cells, respectively. D. Densitometry summary of 4 experiments per group where cells were treated for 24 hours with catalase. *p<0.01 vs untreated control cells;

FIGS. 8A-8B show the effect of dicumarol, alone or in combination with vitamin K, on cell proliferation and NF-κB activation in M1619 cells.

FIGS. 9A-9F show that ferricytochrome c reduction, NF-κB activation, and cellular proliferation of melanoma cells are reduced by dicumarol.

### DETAILED DESCRIPTION OF THE INVENTION

The present invention now will be described more fully hereinafter with reference to the accompanying examples, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “antioxidant” refers to a substance that can significantly delay or prevent oxidation of a biological molecule in a chemical composition or a cell structure containing an oxidizable substrate. As is generally known in the art, antioxidants typically are capable of scavenging or preventing the formation of reactive free radicals or other reactive oxygen species such as .O₂⁻, H₂O₂, .OH, HOCl, ferryl, peroxyl, peroxynitrite, alkoxyl, and the like, or converting them to a less reactive state. There are many antioxidants known in the art including but not limited to enzymes such as catalase, superoxide dismutase, selenium glutathione peroxidase, phospholipid hydroperoxide glutathione peroxidase, glutathione S-transferase, and the like, and organic chemicals such as ascorbic acid, uric acid, tocopherols, tocotrienols, carotenoids, quinones, bilirubin, N-acetylcysteine, allopurinol, dimethyl thiourea, glutathione, ovothiols, pyrazolopyrimidines, butyl-α-phenylnitrone, desferrioxamine and other ion chelators, aminosteroids, N-2-mercaptopropionylglycine, mannitol, etc.

Antioxidants known in the art also include mimetics of various enzymatic antioxidants including catalase mimes, e.g., salen-transition metal complexes disclosed in U.S. Pat. No. 5,696,109, which is incorporated herein by reference, glutathione peroxidase mimes, e.g., ebselen, and the like. Antioxidant also refers to various derivatives of the antioxidant enzymes including muteins and fragments thereof, and conjugates of the enzymes with polymers such as polyethylene glycol.

Suitable antioxidants also include quinone reductase inhibitors, and in particular NAD(P)H:quinone oxidoreductase inhibitors. NAD(P)H:quinone oxidoreductase (NQO, EC 1.6.99.2) is a homodimeric ubiquitous cytosolic and membrane flavoprotein. NQO reduces cellular ubiquinone to ubiquinol, which can redox cycle with molecular oxygen to produce the reactive oxygen species O₂⁻. Thus, by inhibiting NQO, the production of reaction oxygen species O₂⁻ and H₂O₂ is reduced or inhibited. Inhibitors of NQO include, but are not limited to dicumarol, apocynin, diphenylene iodonium chloride, capsaicin, phenprocoumon (4-hydroxy-3-(1-phenylpropyl)-2H-1-benzopyran-2-one), warfarin (3-(α-acetonylbenzyl)-4-hydroxycoumarin), 7-[Diethylamino]-4-trifluoromethylcoumarin, 7-amino-4-trifluoromethylcoumarin, 7-amino-4-methylcoumarin, 4-methylcoumarin, 7-hydroxy-4-methylcoumarin, 7,8-dihydroxy-4-methylcoumarin, 1,1-dimethyl-alloylcoumarin, and the like.

Preferably, the antioxidants used in the present invention are compounds capable of inhibiting the formation of, or catalytically or stoichiometrically removing, reactive oxygen species in mammalian cells. More preferably, the antioxidants used are capable of inhibiting the formation of, or catalytically or stoichiometrically removing hydrogen peroxide. For example, such antioxidants include inhibitors of cellular NAD(P)H:quinone oxidoreductase (NQO, EC 1.6.99.2) (e.g., dicumarol), catalase and derivatives and mimetics thereof, glutathione peroxidase and derivatives and mimetics thereof, N-acetylcysteine, and the like.

The term “treating cancer” as used herein, specifically refers to administering therapeutic agents to a patient diagnosed of cancer, i.e., having established cancer in the patient, to inhibit the further growth or spread of the malignant cells in the cancerous tissue, and/or to cause the death of the malignant cells.

The term “preventing cancer” as used herein, refers to administering a pharmaceutical composition to a patient free of cancer to prevent the formation of cancer and inhibit the transformation of normal cells.

As used herein, “NF-κB” means proteins and protein complexes capable of binding to a consensus DNA sequence of 5′-GGGPuNNPyPyCC-3′ either in vivo or in vitro, and having at least an NF-κB subunit of, e.g., p65, p50, p52, Rel B, and c-Rel, and the like. See Siebenlist et al. Annu. Rev. Cell Biol. 10:405-455 (1994), which is incorporated herein by reference. As is known in the art, typically cellular NF-κB can be detected by, e.g., immunoassays using an antibody specific to one of the NF-κB subunits, or by nuclear protein gel mobility shift assay using an NF-κB binding DNA consensus sequence.

As used herein, the terms “inhibiting the translocation of NF-κB” and “inhibiting activation of NF-κB” are intended to mean that when a chemical agent is applied to a mammalian cell, the translocation of NF-κB, or a subunit thereof or a complex containing NF-κB, from cytoplasm to nucleus is prevented or the amount of NF-κB in cell nucleus is substantially reduced, as compared to the NF-κB in cells to which the chemical agent is not administered. NF-κB activation or translocation can be determined by various methods known in the art. For example, the translocation can be determined quantitatively by histochemical and immunological techniques or qualitatively by gel mobility shift assays to determine the presence or absence of NF-κB, as will be clear from the description below. The translocation of NF-κB is reduced by at least about 10%, preferably at least about 25%, more preferably at least about 50%, and most preferably at least about 70% as compared to that in control cells.

In accordance with one aspect of this invention, a method for treating cancer in a patient is provided including a step of administering to the patient a therapeutically effective amount of an antioxidant. A method for preventing cancer in a patient is also provided including administering to the patient a prophylactically effective amount of an antioxidant.

The present invention also provides a method for inhibiting activation of NF-κB in a mammalian cell by administering to the cell an amount of an antioxidant such that the translocation of NF-κB from cytoplasm to nucleus and the activation of NF-κB are inhibited.

Preferably, an antioxidant selected from the group of catalase, inhibitors of NAD(P)H:quinone oxidoreductase, N-acetylcysteine, glutathione peroxidase, salen-transition metal complexes, and mimes and derivatives thereof is used.

In a preferred embodiment, catalase is administered to a patient for purposes of treating or preventing cancer in the patient. Catalase, i.e., hydrogen-peroxide oxidoreductase (1.11.1.6) is an antioxidant enzyme which scavenges hydrogen peroxide and converts it to water and oxygen. Catalase has been proposed for use in treating various diseases including bums, trauma, stroke, renal transplants, respiratory distress syndrome, broncho-pulmonary displasia, and reperfusion injury following ischemia in myocardial infarction.

Various forms of catalases have been identified and isolated from organisms including animals, plants, fungi and bacteria. Typically, catalase has a molecular weight of about 240 Kilodaltons. Any forms of catalase can be used in the present invention so long as it has catalytic activity of decomposing hydrogen peroxide in mammalian cells. Catalases isolated from animal livers (bovine hepatocatalase) and kidneys as well as bacteria (e.g., Micrococcus Lysodeikticus) and fungi (e.g., Aspergillus Niger) are commercially available and can all be used in the present invention. Catalase from other sources, e.g., produced by genetic engineering, can also be used. In addition, various modified forms or derivatives of catalase can be used. For example, muteins, i.e., mutant forms of catalase containing fragments of catalase or having substituent amino acids in the polypeptide chain, can be useful. Catalase conjugated with polyethylene glycol is especially useful in the present invention because of its reduced immunogenicity and increased stability.

Salen-transition metal complexes can also be administered in the methods of this invention. Salen-transition metal complexes have been shown to have potent antioxidant activities. They are catalase and/or superoxide dismutase mimetics capable of catalyzing the conversion of hydrogen peroxide to water and oxygen. See, e.g., U.S. Pat. No. 5,834,509. Various salen-transition metal complexes are disclosed in, e.g., U.S. Pat. Nos. 5,403,834, 5,696,109, 5,827,880, and 5,834,509, all of which are incorporated herein by reference. All of such salen transition metal complexes can be useful in the present invention. Typically, the salen-transition metal complex has a formula (I): 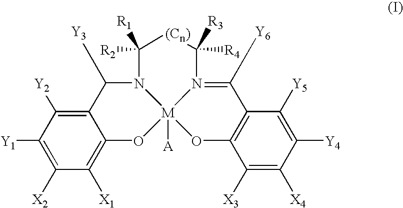
wherein M is a transition metal ion, preferably Mn; A is an anion, typically Cl; and n is either 0, 1, or 2; X₁, X₂, X₃ and X₄ are independently selected from the group consisting of hydrogen, silyls, aryls, arylalkyls, primary alkyls, secondary alkyls, tertiary alkyls, alkoxys, aryloxys, aminos, quaternary amines, heteroatoms, and hydrogen; typically X₁ and X₃ are from the same functional group, usually hydrogen, quaternary amine, or tertiary butyl, and X₂ and X₄ are typically hydrogen; Y₁, Y₂, Y₃, Y₄, Y₅, and Y₆ are independently selected from the group consisting of hydrogen, halides, alkyls, aryls, arylalkyls, silyl groups, aminos, alkyls or aryls bearing heteroatoms; aryloxys, alkoxys, and halide; preferably, Y₁ and Y₄ are alkoxy, halide, or amino groups; typically, Y₁ and Y₄ are the same; R₁, R₂, R₃ and R₄ are independently selected from the group consisting of H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls.

The salen-transition metal complex can also have a formula (II): 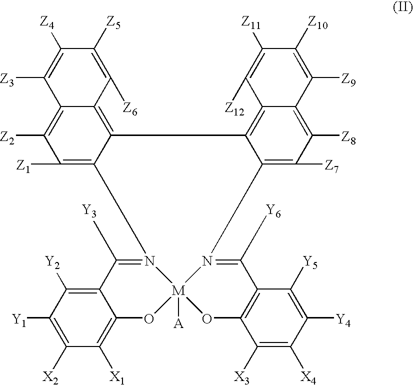
wherein M is a transition metal ion, preferably Mn, and A is an anion, typically Cl; where at least one of X₁ or X₂ is selected from the group consisting of aryls, primary alkyls, secondary alkyls, tertiary alkyls, and heteroatoms; where at least one of X₁ or X₃ is selected from the group consisting of aryls, primary alkyls, secondary alkyls, tertiary alkyls, arylalkyls, heteroatoms, and hydrogen, preferably tertiary butyl or hydrogen; and where Y₁, Y₂, Y₃, Y₄, Y₅, Y₆, Z₁, Z₂, Z₃, Z₄, Z₅, Z₆, Z₇, Z₈, Z₉, Z₁₀, Z₁₁, and Z₁₂ are independently selected from the group consisting of hydrogen, halides, alkyls, aryls, amines, alkoxy, substituted alkoxy, arylalkyls, aryloxys, and alkyl groups bearing heteroatoms. Preferably Y₁, and Y₄ are selected from the group consisting of lower alkyls, alkoxy, halide, and amino groups, more preferably from the group consisting of methoxy, chloro, and primary amine. The salen-transition metal complex can also have a formula (III): 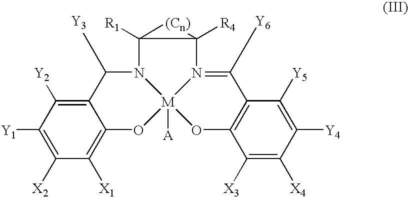
where M is transition metal ion, typically Mn, and A is an anion, typically Cl; where n is either 4, 5, or 6; where X₁, X₂, X₃, and X₄ are independently selected from the group consisting of aryls, arylalkyls, aryloxys, primary alkyls, secondary alkyls, tertiary alkyls, alkoxy, substituted alkoxy, heteroatoms, aminos, quaternary amines, and hydrogen; preferably, at least one of X₁ or X₃ are selected from the group consisting of aryls, primary alkyls, secondary alkyls, tertiary alkyls, quaternary amines, arylalkyls, heteroatoms, and hydrogen; preferably X₁ and X₃ are identical and are hydrogen or tertiary butyl; where Y₁, Y₂, Y₃, Y₄, Y₅, and Y₆ are selected from the group consisting of aryls, arylalkyls, primary alkyls, secondary alkyls, tertiary alkyls, alkoxys, substituted alkoxys, aryloxys, halides, heteroatoms, aminos, quaternary amines, and hydrogen; preferably at least one of Y₁ or Y₄ are selected from the group consisting of aryls, primary alkyls, secondary alkyls, tertiary alkyls, substituted alkoxy, heteroatoms, amines, and halides; more preferably Y₁ and Y₄ are identical and are either methoxy, chloro, bromo, iodo, tertiary butyl, or amine. R₁ and R₄ are independently selected from the group consisting of hydrogen, halides, primary alkyls, secondary alkyls, tertiary alkyls, fatty acid esters, alkoxys, or aryls. Preferably R₁ and R₄ are identical; more preferably R₁ and R₄ are hydrogen.

In a preferred embodiment of the present invention, the salen-manganese complex EUK-8 (Formula IV) or EUK-134 (Formula V) or both are administered to a patient for purposes of treating or preventing cancer. Both EUK-8 and EUK-134 are known in the art and have been studied for its antioxidant activities. See Gonzalez et al. J. Pharmacol. Exp. Ther. 275:798-806 (1995); Baker et al., J. Pharmacol. Exp. Ther. 284:215-221 (1998); and Rong et al., Proc. Natl. Acd. Sci. USA, 96:9897-9902 (1999), all of which are incorporated herein by reference.

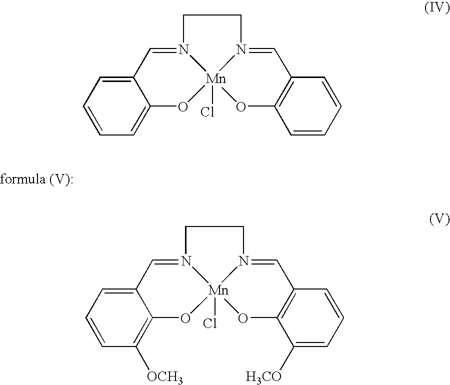

In accordance with the present invention, it has been discovered that organochalcogen compounds such as 2-phenyl-1,2-benzisoselenazol-3(2H)-one (Ebselen), when administered to malignant tumor cells, can effectively inhibit the activation of NF-κB and significantly inhibit the growth of the cells. Accordingly, in another preferred embodiment, one or more organochalcogen compounds known in the art including organoselenium compounds and organotellurium compounds can be administered to a patient for the treatment or prevention of cancer. Preferably, the glutathione peroxidase mimetics 2-phenyl-1,2-benzisoselenazol-3(2H)-one (Ebselen) or an analog thereof is administered.

In accordance with another embodiment of the present invention, dicumarol (3,3′-methylenebis[4-hydroxy-2H-1-benzopyran-2-one]) or an analog thereof is administered in the method of this invention. It has been found that dicumarol is effective in suppressing NAD(P)H:quinnone oxidoreductase activity and inhibiting constitutive activation of NF-κB in tumor cells and can significantly reduce tumor cell growth. Dicumarol has been used clinically for years as an anticoagulant and has been proved to be relatively non-toxic and safe. See Merck Index, 12th Edition, Reference 3140, page 523, Merck & Co., Rahway, N.J., which is incorporated herein by reference. Its chemical properties, and toxicology and pharmacology profiles are well studied and known in the art. See, e.g., Link et al., J. Biol. Chem. 138:21, 513, 529 (1941); J. Biol. Chem., 142:941 (1942); Link, Fed. Proc., 4:176 (1945); Rose et al., Proc. Soc. Exp. Boil. Med., 50:228 (1942), all of which are incorporated herein by reference. Therefore, the use of dicumarol in this invention offers a readily available and easily used treatment for cancer in man and other mammals.

Other types of dicumarol analogs can also be used including phenprocoumon (4-hydroxy-3-(1-phenylpropyl)-2H-1-benzopyran-2-one), warfarin (3-(α-acetonylbenzyl)-4-hydroxycoumarin), 7-[Diethylamino]-4-trifluoromethylcoumarin, 7-amino-4-trifluoromethylcoumarin, 7-amino-4-methylcoumarin, 4-methylcoumarin, 7-hydroxy-4-methylcoumarin, 7,8-dihydroxy-4-methylcoumarin, 1,1-dimethyl-alloylcoumarin, and the like. In particular, like dicumarol, warfarin and phenprocoumon have also been used clinically as anticoagulants.

In accordance with another embodiment of this invention, the method for treating cancer or inhibiting malignant cell growth includes administering to the patient or contacting tumor cells with dicumarol (or an analog thereof) and vitamin K (or an analog or derivative thereof). Dicumarol is known in the art as an anticoagulant which acts by depressing Factors VII, IX, X, and II which are active in the coagulation mechanism. Dicumarol has been produced by Abbott Laboratories and used in the prophylaxis or treatment of venous thrombosis, atrial fibrillation with embolization, pulmonary embolism, and coronary occlusion. See Physician's Desk Reference, 44^{th} Ed., 1990, at page 518. In accordance with the present invention, it has been discovered that addition of equimolar concentration of dicumarol and vitamin K does not impair the growth inhibiting effect of dicumarol. That is, inhibition of oxidant signaling of NF-κB activation and tumor cell growth by dicumarol is not materially caused by interfering with a previously unrecognized aspect of vitamin K metabolism. Therefore, vitamin K can be administered to a patient to offset the anticoagulant effect of dicumarol without adversely affecting the anticancer effect of dicumarol. For this purpose, “vitamin K” means any member of the vitamin K group, i.e., the group of naphthoquinone derivatives required for the bioactivation of proteins involved in hemostasis. Commonly known vitamin K members include, but are not limited to, phylloquinone (vitamin K₁), menaquinones (vitamin K₂), menadione (vitamin K₃), and vitamin K₅, and the like. “Vitamin K” also means any derivatives and analogs of the vitamin K group members having similar functions in homeostasis, including but not limited to, dihydrovitamin K, menaquinone-4, and derivatives and analogs thereof.

Dicumarol can be administered simultaneously in the same pharmaceutical preparation with vitamin K. Dicumarol and vitamin K can also be administered at about same time but by a separate administration. Alternatively, dicumarol can be administered at a different time from the administration of vitamin K. Some minor degree of experimentation may be required to determine the best manner of administration, this being well within the capability of one skilled in the art once apprised of the present disclosure. Preferably, vitamin K and dicumarol are applied at about the same time to a patient needing treatment in the same or different pharmaceutical compositions.

In addition, various NAD(P)H oxidase inhibitors can also be administered to tumor cells to inhibit the malignant growth of the cells and to treat cancer. Examples of suitable inhibitors include, but are not limited to, quinone analogs such as capsaicin, diphenylene iodonium chloride, and apocynin.

In accordance with another aspect of this invention, the method of this invention can be used in combination with a conventional anticancer therapy. For example, the method of this invention can be complemented by a conventional radiation therapy or chemotherapy. Thus, in one embodiment of this invention, the method of this invention comprises administering to a patient an antioxidant as described above and another anticancer agent.

Any anticancer agents known in the art can be used in this invention so long as it is pharmaceutically compatible with the antioxidant compounds used. By “pharmaceutically compatible” it is intended that the other anticancer agent will not interact or react with the above composition, directly or indirectly, in such a way as to adversely affect the effect of the treatment of cancer, or to cause any significant adverse side reaction in the patient.

Exemplary anticancer agents known in the art include cisplatin, carmustine, herceptin, carboplatin, cyclophosphamide, nitrosoureas, fotemustine, vindesine, etoposide, daunorubicin, adriamycin, taxol, taxotere, fluorouracil, methotrexate, melphalan, bleomycin, salicylates, aspirin, piroxicam, ibuprofen, indomethacin, maprosyn, diclofenac, tolmetin, ketoprofen, nabumetone, oxaprozin, doxirubicin, nonselective cyclooxygenase inhibitors such as nonsteroidal anti-inflammatory agents (NSAIDS), and selective cyclooxygenase-2 (COX-2) inhibitors.

The anticancer agent used can be administered simultaneously in the same pharmaceutical preparation with the antioxidant compound as described above so long as they are pharmaceutically compatible and no adverse effect is caused. The anticancer agent can also be administered at about same time but by a separate administration. Alternatively, the anticancer agent can be administered at a different time from the administration of the antioxidant compound. Some minor degree of experimentation may be required to determine the best manner of administration, this being well within the capability of one skilled in the art once apprised of the present disclosure.

The methods of this invention are suitable for treating cancers in animals, especially mammals such as canine, bovine, porcine, and other animals. Advantageously, the methods are used in treating human patients. The methods are useful for treating various types of cancer, including but not limited to melanoma, non-small cell lung cancer, small cell lung cancer, renal cancer, colorectal cancer, breast cancer, pancreatic cancer, gastric cancer, bladder cancer, ovarian cancer, uterine cancer, lymphoma, and prostate cancer. In particular, the present invention will be especially effective in treating melanoma, lung cancer, breast cancer, and prostate carcinoma.

The active compounds of this invention are typically administered in a pharmaceutically acceptable carrier through any appropriate routes such as parenteral, intravenous, oral, intradermal, subcutaneous, or topical administration. The active compounds of this invention are administered at a therapeutically effective amount to achieve the desired therapeutic or prophylactic effect without causing any serious adverse effects in the patient treated.

The dosage and pharmaceutical formulation of these compounds developed in treating other diseases can be equally effective and applicable to the anticancer treatment of the present invention. For other active compounds, the therapeutically or prophylactically effective dosage ranges can be readily determined empirically using known testing protocols or by extrapolation from in vivo or in vitro test data as will be apparent to skilled artisans.

Generally speaking, the broad dosage range of the active compounds used in the method of the present invention for effective treatment of cancer is about 0.001 to 100 milligram (mg) per kilogram (kg) of body weight of the patient per day. The preferred range is about 0.01 to 10 mg/kg of body weight per day.

For example, dicumarol can be effective when administered at an amount within the conventional clinical ranges determined in the art. Typically, it can be effective at an amount of from about 0.1 mg to about 5000 mg per day, preferably from about 10 mg to about 1000 mg per day. The suitable dosage unit for each administration of dicumarol can be, e.g., from about 5 to about 1000 mg, preferably from about 25 to about 500 mg. Dicumarol has been used as anticoagulant and its toxicology data is disclosed in, e.g., Rose et al., Proc. Soc. Exp. Boil. Med., 50:228 (1942), with an LD₅₀ (orally in rats) of 541.6 mg/kg. Dicumarol can be available from Abbott Laboratories in tablet forms. See Physician's Desk Reference, 44^{th} Ed., 1990, at page 518. When the treatment includes administering vitamin K and dicumarol, the dosage of vitamin K can be within the conventional dosage ranges as known in the art. For example, vitamin K can be used in a dosage of about 0.1 mg to about 500 mg per day, preferably from about 0.5 mg to about 200 mg per day.

EUK-8 and EUK-134 can be administered at a dosage of from about 0.01 mg/kg to about 100 mg/kg per day, preferably from about 0.1 mg/kg to about 10 mg/kg per day, and more preferably from about 1 mg/kg to about 5 mg/kg per day based the total body weight of the patient. The suitable dosage unit for each administration of EUK-8 or EUK-134 can be, e.g., from about 50 to about 1000 mg, preferably from about 250 to about 500 mg.

The suitable dosage unit for each administration of ebselen can be, e.g., from about 5 to about 1000 mg, preferably from about 25 to about 500 mg.

However, it should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, inactivation, and excretion of the active compound by the body, the age and sensitivity of the patient to be treated, and the like, as will be apparent to a skilled artisan. The amount of administration can also be adjusted as the various factors change over time and according to the individual need and/or the professional judgment of the person administering or supervising the administration of the compositions.

The active compounds of this invention can be administered to a patient to be treated through any suitable routes of administration.

Advantageously, the active compounds are delivered to the patient parenterally, i.e., intravenously or intramuscularly. For parenteral administration, the active compounds can be formulated into solutions or suspensions, or in lyophilized forms for conversion into solutions or suspensions before use. Sterile water, physiological saline, e.g., phosphate buffered saline (PBS) can be used conveniently as the pharmaceutically acceptable carriers or diluents. Conventional solvents, surfactants, stabilizers, pH balancing buffers, anti-bacteria agents, and antioxidants can all be used in the parenteral formulations, including but not limited to acetates, citrates or phosphates buffers, sodium chloride, dextrose, fixed oils, glycerin, polyethylene glycol, propylene glycol, benzyl alcohol, methyl parabens, ascorbic acid, sodium bisulfite, and the like. The parenteral formulation can be stored in any conventional containers such as vials, ampoules, and syringes.

The active compounds can also be delivered orally in enclosed gelatin capsules or compressed tablets. Capsules and tablets can be prepared in any conventional techniques. For example, the active compounds can be incorporated into a formulation which includes pharmaceutically acceptable carriers such as excipients (e.g., starch, lactose), binders (e.g., gelatin, cellulose, gum tragacanth), disintegrating agents (e.g., alginate, Primogel, and corn starch), lubricants (e.g., magnesium stearate, silicon dioxide), and sweetening or flavoring agents (e.g., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules.

Other forms of oral formulations such as chewing gum, suspension, syrup, wafer, elixir, and the like can also be prepared containing the active compounds used in this invention. Various modifying agents for flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered topically through rectal, vaginal, nasal or mucosal applications. Topical formulations are generally known in the art including creams, gels, ointments, lotions, powders, pastes, suspensions, sprays, and aerosols. Typically, topical formulations include one or more thickening agents, humectants, and/or emollients including but not limited to xanthan gum, petrolatum, beeswax, or polyethylene glycol, sorbitol, mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

The active compounds can also be delivered by subcutaneous implantation for sustained release. This may be accomplished by using aseptic techniques to surgically implant the active compounds in any suitable formulation into the subcutaneous space of the anterior abdominal wall. See, e.g., Wilson et al., J. Clin. Psych. 45:242-247 (1984). Sustained release can be achieved by incorporating the active ingredients into a special carrier such as a hydrogel. Typically, a hydrogel is a network of high molecular weight biocompatible polymers, which can swell in water to form a gel like material. Hydrogels are generally known in the art. For example, hydrogels made of polyethylene glycols, or collagen, or poly(glycolic-co-L-lactic acid) are suitable for this invention. See, e.g., Phillips et al., J. Pharmaceut. Sci. 73:1718-1720 (1984).

The active compounds can also be conjugated, i.e., covalently linked, to a water soluble non-immunogenic high molecular weight polymer to form a polymer conjugate. Advantageously, such polymers, e.g., polyethylene glycol, can impart solubility, stability, and reduced immunogenicity to the active compounds. As a result, the active compound in the conjugate when administered to a patient, can have a longer half-life in the body, and exhibit better efficacy. PEGylated proteins are currently being used in protein replacement therapies and for other therapeutic uses. For example, PEGylated adenosine deaminase (ADAGEN7) is being used to treat severe combined immunodeficiency disease (SCIDS). PEGylated L-asparaginase (ONCAPSPAR7) is being used to treat acute lymphoblastic leukemia (ALL). For a general review of PEG-protein conjugates with clinical efficacy. See, e.g., Burnham, Am. J. Hosp. Pharm., 15:210-218 (1994). Preferably, the covalent linkage between the polymer and the active compound is hydrolytically degradable and is susceptible to hydrolysis under physiological conditions. Such conjugates are known as “prodrugs” and the polymer in the conjugate can be readily cleaved off inside the body, releasing the free active compounds.

Alternatively, other forms controlled release or protection including microcapsules and nanocapsules generally known in the art, and hydrogels described above can all be utilized in oral, parenteral, topical, and subcutaneous administration of the active compounds.

Another preferable delivery form is using liposomes as carrier. Liposomes are micelles formed from various lipids such as cholesterol, phospholipids, fatty acids, and derivatives thereof. Active compounds can be enclosed within such micelles. Methods for preparing liposomal suspensions containing active ingredients therein are generally known in the art and are disclosed in, e.g., U.S. Pat. No. 4,522,811, which is incorporated herein by reference. Several anticancer drugs delivered in the form of liposomes are known in the art and are commercially available from Liposome Inc. of Princeton, N.J., U.S.A. It has been shown that liposomal can reduce the toxicity of the active compounds, and increase their stability.

The active compounds can also be administered in combination with other active agents that treats or prevents another disease or symptom in the patient treated. However, it is to be understood that such other active agents should not interfere with or adversely affect the effects of the active compounds of this invention on the cancer being treated. Such other active agents include but are not limited to antiviral agents, antibiotics, antifungal agents, anti-inflammation agents, antithrombotic agents, cardiovascular drugs, cholesterol lowering agents, hypertension drugs, and the like.

### EXPERIMENTAL MATERIALS AND METHODS

Materials. Human malignant cell lines were obtained from American Type Tissue Culture Collection (Rockville, Md.). RPMI medium 1640, Leibovitz=s L-15 medium, N-2-hydroxyethylpiperazine-N=-2-ethanesulfonic acid (HEPES), antibiotic-antimycotic (10,000 U penicillin, 10,000 μg streptomycin, and 25 μg amphotericin B/ml), and trypsin-ethylenediaminetetraacetic acid (EDTA) solution were purchased from the GIBCO-BRL division of Life Technologies (Grand Island, N.Y.). Fetal bovine serum was purchased from HyClone Laboratories (Logan, Utah). Rabbit polyclonal antibodies for protein immunoassay and supershift antibodies for electrophoretic mobility shift assays (EMSAs) were purchased from Santa Cruz Biotechnology (Santa Cruz, Calif.), with the exception of phospho-specific antibodies detecting the phosphorylated form of IκBα, which were purchased from New England Biolabs (Beverly, Mass.). Peroxidase-labeled donkey polyclonal anti-rabbit IgG was from Amersham Life Sciences (Buckinhamshire, England). EMSA supplies, including DNA probes, were purchased from ProMega (Madison, Wis.). Protease inhibitors were from Boehringer Mannheim (Indianapolis, Ind.). All other materials were purchased from Sigma Chemical Co. (St. Louis, Mo.), unless specified.

Culture of Malignant Cell Lines. Melanoma cell lines CRL 1585 and 1619 were cultured in RPMI 1640 with 10% FBS and passed with non-enzymatic Cell Dissociation Solution7 (Sigma). LNCaP.FGC prostate adenocarcinoma cells were also cultured in RPMI 1640 with 10% FBS but passed with 0.05% trypsin and 0.53 mM EDTA. The adenosquamous lung carcinoma NCI-H596 cell line was grown in RPMI 1640 supplemented with 10% FBS, 10 mM HEPES and 1.0 mM sodium pyruvate and passed with trypsin/EDTA. All of the above were grown in a 37° C. humidified environment containing 5% CO₂/air. The breast carcinoma cell line MDA-MB-453 was grown in a 37° C. humidified environment with free gas exchange with atmospheric air using Leibovitz's L-15 medium with 2 mM L-glutamine and 10% FBS and was passed with trypsin/EDTA.

Measurement of Proliferation in Cell Cultures. Proliferation of cultured cells was quantitated using a previously reported calorimetric method based upon metabolic reduction of the soluble yellow tetrazolium dye 3-[4,5-dimethylthiazol]-2yl-2,5-diphenyl tetrazolium bromide (MTT) to its insoluble purple formazan by the action of mitochondrial succinyl dehydrogenase. See Brar et al., J. Biol. Chem., 274:20017-20026 (1999). This assay empirically distinguishes between dead and living cells. For proliferation studies, cells were seeded into 24-well uncoated plastic plates (Costar) at 15,000-50,000 cells per well and cultured with respective media and mitogens. After 24-96 hours, medium was removed, cells were washed twice with 1 ml of sterile Dulbecco's modified phosphate buffered saline without Ca²⁺ or Mg²⁺ (DPBS), the medium was replaced with 1 ml/well fresh medium containing 100 μg/ml MTT, and plates were incubated an additional hour. MTT-containing medium was removed, 0.5 ml dimethylsulfoxide (DMSO) was added to each well, and the absorbance of the solubilized purple formazan dye was measured at 540 nm. A total of 4-6 wells was studied at each treatment condition. Preliminary studies were performed with 50-200 μg/ml MTT incubated for 15 min to 3 hours to determine the optimum concentration and incubation time at which the rate of conversion was linear and proportional to the number of cells present. The absorbance of the MTT formazan reduction product (A₅₄₀) correlated with cell numbers counted by hemocytometer with an R²=0.99. In some experiments, the MTT assay and responses to mitogens and inhibitors were also confirmed by performing cell counts on 10 random fields/well of Giemsa-modified Wright's stained monolayers viewed at 40 power using a 0.01-cm² ocular grid.

Cell Culture Treatments. The effect of antioxidant treatments on proliferation of malignant cell lines was studied in cultures stimulated with 10% FBS. Cell numbers were quantitated by the MTT assay 24-72 hours later. In some experiments, antioxidants were added immediately after cells were plated. In other experiments, cells were plated and allowed to grow for 24 hours before fresh media with antioxidants was added, and cell numbers were studied by the MTT assay 48 hours later.

The effect of antioxidants on nuclear activation of NF-κB was studied by incubating near confluent (70%) cell cultures with antioxidant treatments for 1-48 hours. Nuclear protein was harvested, and EMSAs were performed using DNA consensus binding sequences, or nuclear translocation of the p65 component of NF-κB was studied by immunoperoxidase staining, as outlined below.

The effect of antioxidants on expression of cell cyclins, cyclin-associated kinases, cell cycle inhibitors and IκBα was studied by incubating near confluent cell cultures with antioxidant treatments for 15 min-48 hours. Cells were lysed and protein levels were measured by immunoblot assay as described below. In other experiments, the ratio of phosphorylated-IκBα to total IκBα was determined by harvest of cytosolic protein 24 hours after treating cultures with 3,000 U/ml catalase.

To study the impact of inhibiting NF-κB activation with antioxidants, cellular expression of the autocrine growth factor GRO-α was studied in near confluent monolayers of M1619 cells grown in 24-well plates. Cells were either treated with fresh complete medium or fresh medium containing 25 μM ebselen. After 24 hours, supernatants were harvested, microcentrifuged to remove cellular debris and frozen at −20° C. until GRO-α measurement as outlined below.

Measurement of Cytotoxicity and Apoptosis. To assess for cytotoxicity, near confluent cells cultured in 24 well plates were exposed to antioxidants or withdrawn from serum for 24 hours. Medium was removed, and replaced with Dulbecco=s phosphate buffered saline (DPBS) containing 0.1% trypan blue. Cell death was assessed by counting the average number of trypan blue positive cells in 5 random fields counted of 8 separate wells at 40 power using a 0.01-cm² ocular grid. To assess for apoptosis, cells grown on glass slides were treated with antioxidants for 24 hours. Apoptosis was studied by terminal deoxynucleotidyl transferase (TdT) dependent 3′BOH fluorescein end-labeling of DNA fragments, using a Fluorescein-FragEL™ DNA fragmentation detection kit (Oncogene Research Products, Cambridge, Mass.). DNase-treated fixed cells were used as a positive control.

DNA Cell Cycle Measurements. To study the effect of antioxidant treatments on the DNA cell cycle, cells were grown to near confluence in 25 cm² plastic flasks and treated for 24 hours. Cells were typsinized, washed twice in cold DPBS with 1 mM EDTA and 1% bovine serum albumin (BSA), fixed 30 min in ice-cold 70% ethanol, and stained by incubation for 30 min at 37° C. in a 10 μg/ml solution of propidium iodide in DPBS and 1 mg/ml RNase A. DNA cell cycle measurements were made using a FACStar^{PLUS} Flow Cytometer (Becton-Dickenson, San Jose, Calif.).

Measurement of Reactive Oxygen Species. Superoxide (O₂⁻) generation was measured by the technique of superoxide dismutase (SOD) inhibitable reduction of ferricytochrome c, employing a modification allowing absorbance reading with an automatic enzyme immunoassay reader. See Friovich, I. in Handbook of Methods for Oxygen Radical Research (Greenwald, R. A., ed.), pp 121-122, CRC Press, Boca Raton, Fla., 1985; Pick, E. et al. J. Immunol. Methods 46:211-226 (1981). Confluent cells grown on 24-well plates were washed with DPBS, and incubated in 5% CO₂/air at 37° C. with 160 μM ferricytochrome c in total volume of 550 μl of sodium bicarbonate-containing Krebs-Heinseleit buffer, or Hanks Balanced Salt Solution (HBSS), with and without copper-zinc SOD (1,000 units/ml). See Nozik-Grayck, et al., Am J. Physiol. 273 (17):L296-L304 (1997). The absorbance of each well was measured at 550 nm initially and 3-24 hours later using an ELx800 UV automated microplate reader (Biotek Instruments, Highland Park, Vt.). Monolayers were then washed with DPBS, and cell protein was measured using the BCA protein assay (Pierce). O₂⁻ generation, normalized to cell protein, was computed from the Beer-Lambert relationship, as the quotient of SOD-inhibitable increase absorbance over time divided by the difference between the molar extinction coefficients for ferricytochrome c and ferrocytochrome c (ΔEM=2.1×10⁴ M⁻¹ cm⁻¹). See Bashford, in Spectrophotometry & Spectrofluorimetry. A Practical Approach (Harris, D. A., and Bashford, C. L., eds) pp. 1-22, IRL Press, Washington, D.C., 1987; Friovich, I. in Handbook of Methods for Oxygen Radical Research (Greenwald, R. A., ed) pp 121-122, CRC Press, Boca Raton, Fla., 1985. In some experiments, the following inhibitors of major oxidases were added to dissect potential sources of O₂⁻ generation: the quinone analog capsaicin (8-methyl-N-vanillyl-6-noneamide, 100 μM), the NAD(P)H:quinone oxidoreductase inhibitor dicumarol (250 μM), the xanthine oxidase inhibitor allopurinol (1 mM), the cyclooxygenase inhibitor indomethacin (10 μg/ml), the cytochrome P450 inhibitor cimetidine (300 μM), the nitric oxide synthase inhibitor N_{ω}-nitro-L-arginine (LNAME, 100 μM) and the mitochondrial respiratory chain inhibitor rotenone (2 μM).

In other experiments, production of H₂O₂ by cell cultures was measured by the phenol red method of Pick and Keisari, adapted for use with an automatic enzyme immunoassay reader. Cultures were incubated at 37° C. with 400 μl of phenol red solution containing 140 mM NaCl, 10 mM potassium phosphate buffer, pH 7.0, 5.5 mM dextrose, 0.56 mM phenol red and 9 U/ml of horseradish peroxidase. At the end of incubation, the reaction was stopped by adding 10 μl 1.0 N NaOH. Absorbance was read at 600 nm and concentration of H₂O₂ was determined by comparison with a standard curve constructed using 0-10 μM H₂O₂.

Determination of oxidase activities and levels of oxidase components. Xanthine dehydrogenase/oxidase (XDH/XO) activity was measured using the spectrofluorometric assay described by Beckman et al., Free Rad. Biol. Med. 6:607 (1989). Briefly, monolayers were washed twice in ice cold DPBS, scraped and frozen in liquid nitrogen. The cell pellet was sonicated in 1 ml of buffer containing 0.1 mM EDTA, 10 mM dithiothreitol (DTT) and 1% (3-[(3-cholamidopropyl) dimethylammonio]-1-propane sulfonate) (CHAPS) in 50 mM phosphate buffer, pH 7.4. The cell lysates were centrifuged at 18,000 g for 30 min at 4° C. The supernatant was diluted to 2 ml with 50 mM phosphate buffer containing 0. 1 mM EDTA, pH 7.4. Fluorescence was monitored at 390 nm with the excitation wavelength set at 345 nm. After achieving a stable baseline, 20 μl of 1 mM pterin was added and the reaction was observed for 20 min to assay XO activity. Subsequently, 20 μl of 1 mM methylene blue was added as an electron accceptor to assay total XDH/XO activity and the reaction was observed for 20 min. To probe for presence of p22 and gp91^{phox} components to the putative analog of neutrophil NAD(P)H oxidase and the newly described mox-1, total RNA was isolated from cells by the method of Chomzynski and Sacchi. See Suh et al., Nature 401:79-82 (1999); Chomczynski, P., and Sacchi, N. Anal. Biochem. 162:156-159 (1987). The RNA concentration was determined spectrophotometrically, and 5 μg were used for reverse transcription employing a standard protocol with Moloney murine leukemia virus reverse transcriptase. Excess RNA was digested with 2 μg DNAse free RNAse (Boehringer Mannheim) and incubated at 37° C. for 5 min. The reaction was extracted with phenol/chloroform and precipitated with ethanol at −20° C. overnight. The cDNA concentration was spectrophotometrically determined. Semi-quantitative PCR was performed by using a known amount of cDNA per reaction and analyzing the radioactive product on a polyacrylamide gel. Optimal cDNA amplification and number of cycles for amplification were determined by titration from 1 to 500 ng of cDNA and from 18 to 40 cycles. Optimal parameters were determined to be 200 ng of cDNA for 20 cycles. PCR buffer containing Mg²⁺ (Perkin-Elmer) and dNTP concentrations of 100 μM were used plus 0.25 μCi of [³²P]dCTP. For consistency of samples, a master mix for each set of primers was prepared. Reactions of 25 μl were amplified, and the PCR conditions were as follows: denaturation at 94° C. for 15 s; annealing for 15 s at 57° C. for gp91^{phox}, at 59° C. for p22 and at 61° C. for gp91^{mox}; and elongation at 72° C. for 30 s. Following PCR an aliquot was added to an equal volume of DNA sample buffer, heated to 95° C. for 5 min, and electrophoresed in a 6% acrylamide gel. Bands were detected by autoradiographic exposure and compared with each other and against amplified β-actin as an internal control. The following specific primer pairs were employed: p22-5′ ATGGAGCGCTGGGGACAGAAGCACATG; p22-3′ GATGGTGCCTCCGATCTGCGGCCG; gp91^{phox}-5′ TCAATAATTCTGATCCTTATTCAG; gp91^{phox}-3′ TGTTCACAAACTGTTATATTATGC; mox-1-5′ AGCAAGAAGCCGACAGGCCACAGAT; mox-1-3′ ACATCTCAAAACACTCTGCACACT; NOH-1L-5′-GCTCCAAACCACCTCTTGAC; and NOH-1L-3′-TGCAGATTACCGTCCTTATTCC.

Electrophoretic Mobility Shift Assays (EMSAs). Nuclear protein was isolated and DNA binding reactions were performed as previously described in detail, Kennedy et al., Am. J. Respir. Cell Mol. Biol. 19:366-378 (1998). Monolayers were washed twice in cold DPBS and equilibrated 10 min on ice with 0.7 ml cold cytoplasmic extraction buffer, CEB (10 mM Tris, pH 7.9, 60 mM KCl, 1 mM EDTA, 1 mM DTT) with protease inhibitors, PI (1 mM Pefabloc, 50 μg/ml antipain, 1 μg/ml leupeptin, 1 μg/ml pepstatin, 40 μg/ml bestatin, 3 μg/ml E-64 and 100 μg/ml chymostatin). The detergent Nonidet P-40 (NP-40) was added to a final concentration of 0.1% and cells were dislodged with a cell scraper. Nuclei were pelleted by centrifugation and washed with CEB/PI. Nuclei were then incubated for 20 min on ice in nuclear extraction buffer, NEB (20 mM Tris, pH 8.0, 400 mM NaCl, 1.5 mM MgCl₂, 1.5 mM EDTA, 1 mM DTT and 25% glycerol) with PI, spun briefly to clear debris and stored at −80° C. until performance of electrophoretic mobility shift assays. EMSAs were performed using the consensus binding oligonucleotides, 5′-AGTTGAGGGGACTTTCCCAGGC-3′ and 3′-TCAACTCCCCTGAAAGGGTCCG-5′, for the p50 component of NF-κB(ProMega, Madison, Wis.), end-labeled by phosphorylation with [γ³²P]-ATP and T4 polynucleotide kinase. DNA-protein binding reactions were performed with 2 μg of nuclear protein (as determined by the Pierce method) and 30-80,000 cpm of ³²P-end-labelled double-stranded DNA probe in 10 mM Tris-HCl, pH 7.5, 50 mM NaCl, 0.5 mM EDTA, 0.5 mM DTT, 1 mM MgCl₂, 50 μg/ml poly dI-dC, and 4% glycerol. All components of the binding reaction with the exception of labeled probe were combined and incubated at room temperature for 10 min before addition of labeled probe and incubation for an additional 20 min. Competition experiments were performed with 10× unlabeled wild-type oligonucleotide sequences for NF-κB added before labeled probe. Supershift assays were performed by adding 1.0 μg of supershift-specific antibodies for p65, p50, p52, Rel B, or c-Rel components of NF-κB and incubating at room temperature for 30 min or overnight at 4° C. before adding the probe. Samples were electrophoresed on a 5% nondenaturing polyacrylamide gel in Tris-glycine-EDTA (TGE, 120 mM glycine and 1 mM EDTA in 25 mM Tris, pH 8.5) buffer. Gels were dried and analyzed by autoradiography at −80° C. using an image intensifier screen. Densitometry of bands was performed using Kodak Digital Science 1D image analysis software (Eastman Kodak, Rochester, N.Y.).

Immunohistochemical Localization of NF-κB. Cells grown on sterile coverslips and treated with catalase 3,000 U/ml, apocynin 150 μg/ml, or DPBS or DMSO vehicles for 24 h were fixed for 20 min on ice with 4% paraformaldehyde in DPBS with protease inhibitors, PI [Sigma protease inhibitor cocktail, 10 μl/ml, containing 4-(2-aminoethyl)benzensulfonyl fluoride (AEBSF), pepstatin A, trans-epoxysuccinyl-L-leucylamindo-(guanidino)butane (E-64), bestatin, leupeptin and aprotinin]. Cells were permeabilized by treating for 2 min with 0.1% NP-40 in DPBS/PI, washed once with cold DPBS and fixed as before for 10 min. Coverslips were incubated in 3% hydrogen peroxide for 30 min to suppress any remaining peroxidase, and washed three times in cold DPBS. The permeabilized and fixed cells were blocked for 2 hours with 2% BSA in DPBS on ice and incubated overnight at 4° C. with 1 μg/ml of anti-p65 antibody (Santa Cruz) diluted in 0.1% BSA/DPBS. Unbound anti-p65 was washed away with 2% BSA/DPBS and bound antibody was stained by incubation with biotinylated goat anti-rabbit immunoglobulin diluted 1:50 in 0.1% BSA/DPBS for 45 min on ice. Excess secondary antibody was washed away by 3 washes with 2% BSA/DPBS on ice. After washing, the cells were incubated with a streptavidin-biotin-peroxidase complex at room temperature for 1 hr, washed again, and incubated in 0.03% wt/vol 3-3′diaminobenzidine with 0.003% vol/vol hydrogen peroxide until a brown reaction product could be seen. Cells were then counterstained with eosin and mounted on glass slides before viewing under light microscopy.

Immunoassay for Proteins. Cells were lysed and proteins were isolated and quantitated by immunoassay as previously detailed, using 2 μg/ml of primary rabbit polyclonal antibodies against human p53, the cyclins D1, E, A and B1, the cyclin-associated kinases cdk2 and cdc2, the cyclin kinase inhibitors p21^{WAF1/Cip1} and p27, and peroxidase-labeled donkey polyclonal anti-rabbit IgG. Brar et al., J. Biol. Chem. 274:20017-20026 (1999). Cells were placed on ice, washed twice with cold DPBS, scraped into 0.5 ml boiling buffer (10% [vol/vol] glycerol and 2% [wt/vol] sodium dodecyl sulfate [SDS] in 83 mM Tris, pH 6.8) and sheared by four passages through a pipette. Aliquots were removed for protein determination, using the BCA protein assay (Pierce). After 10% β-mercaptoethanol and 0.05% bromophenol blue were added, lysates were boiled for 5 min and stored at −80° C. until immunoblotting was performed. Proteins in defrosted samples were separated by SDS-polyacrylamide gel electrophoresis on 12% polyacrylamide gels (15 μg protein/lane) and electrotransferred to 0.45 μm Hybond ECL nitrocellulose membranes (Amersham Life Sciences) using the wet transblot method in transfer buffer (0.025 M Tris, 0.192 M glycine, 2.6 mM SDS, and 20% [vol/vol] methanol; pH 8.8) at 100 volts for 1 hour. Blots were blocked overnight at 4° C. with blocking buffer (PBS with 0.1% Tween 20) containing 5% fat-free milk powder (Carnation, Glendale, Calif.). After rinsing 5 times for 5 min each in PBS containing 0.1% Tween 20, blots were incubated for 1 hour at room temperature with 2.0 μg/ml of primary antibody. After rinsing again as above, blots were incubated for 1 hour at room temperature with horseradish peroxidase(HRP)-conjugated secondary antibody diluted 1:5,000 in blocking buffer. Immunoblots were rinsed again as above and detected using an enhanced chemiluminescence method (ECL Western blotting detection system, Amersham Life Science, Buckinghamshire, England) and autoradiography. Densitometry was performed as above.

The NF-κB inhibitor IκBα and phosphorylated IκBα were assayed with several modifications of the above procedure. Cells were lysed in boiling buffer to which 50 mM DTT had been added as a reducing agent. Immunoassay of IκBα. proceeded as above, but samples for measurement of phosphorylated IκBα were blocked 2 hours at room temperature and incubated overnight at 4° C. with primary phospho-specific antibodies diluted 1:1000 in PBS with 0.1% Tween and 5% BSA.

Measurement of GRO-α Expression. GRO-α was measured in culture medium from untreated and ebselen treated cells using a commercial ELISA purchased from R&D Systems, Minneapolis, Minn. This assay could not be used to detect GRO-α production by catalase treated cells because of interference in the peroxidase-based ELISA by catalase in the cell supernatant.

Statistical Analysis. Data are expressed as mean values ∀ standard error. The minimum number of replicates for all measurements was four, unless indicated. Differences between two groups were compared using the Student=s t test. Differences between multiple groups were compared using one-way analysis of variance. The post-hoc test used was the Newman-Keuls multiple comparison test. Two-tailed tests of significance were employed. Significance was assumed at p<0.05.

### EXPERIMENT 1

Antioxidants are antiproliferative against malignant human cell lines.

A. M1619 melanoma cells stimulated with 10% fetal bovine serum (FBS) were plated at a density of 50,000 cells per well and antioxidants were added to wells at the indicated concentrations (mM or U/ml). After 48 hours, proliferation was quantitated by assessing the cell number-dependent reduction of the soluble yellow tetrazolium dye 3-[4,5-dimethylthiazol]-2yl-2,5-diphenyl tetrazolium bromide (MTT) to its insoluble formazan, measured as the absorbance at 540 nm (A₅₄₀). The antioxidants tested included no antioxidant (control, i.e., FBS alone), 1000 U/ml SOD, 20 mM NAC, 500 U/ml, 1000 U/ml, and 3000 U/ml of catalase, and boiled 3000 ml catalase.

B. In a separate experiment, 2 μl/well of DMSO (served as control), 5 μM, 10 μM, and 25 μM Ebselen were tested on M1619 melanoma cells as described above, except that the cells were cultured for 72 hours before proliferation was measured.

C. 3000 U/ml Catalase, 2 μl/well DMSO, 25 μM Ebselen, and 50 μM Ebselen were tested as described above except that the compounds were added 24 hours after M1619 cells were plated. After the addition of antioxidants, the cells were incubated for an additional 48 hours and cell proliferation was quantitated.

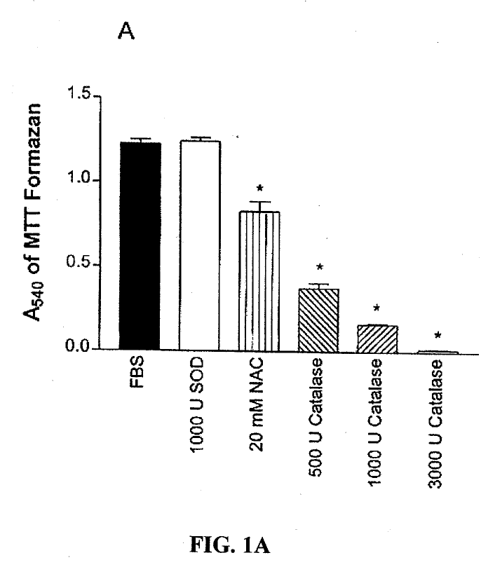

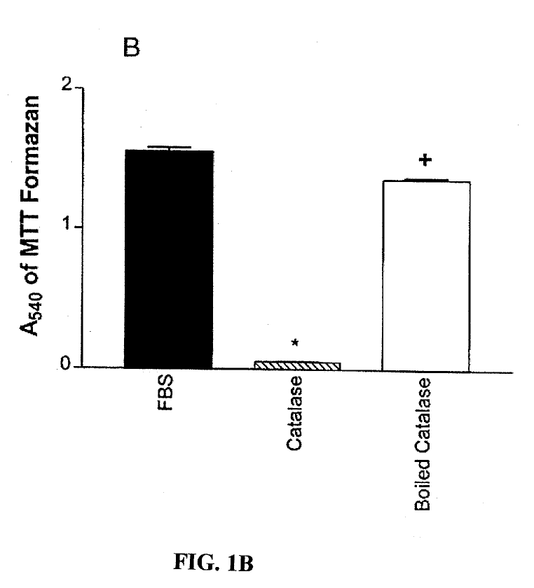

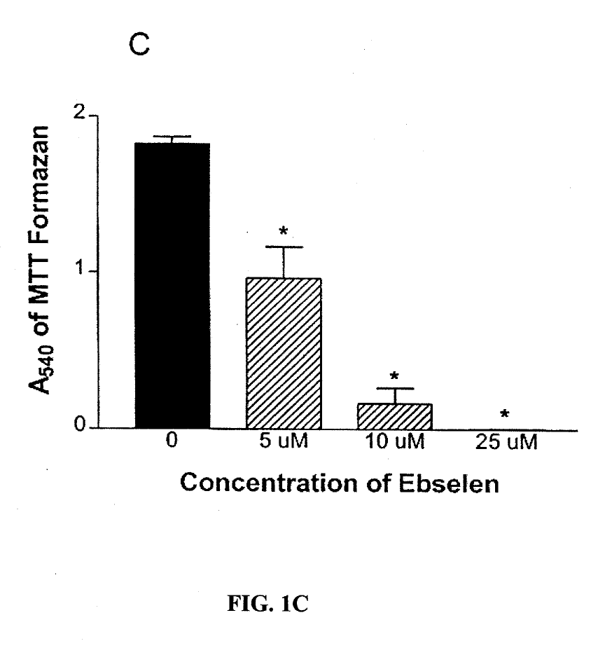

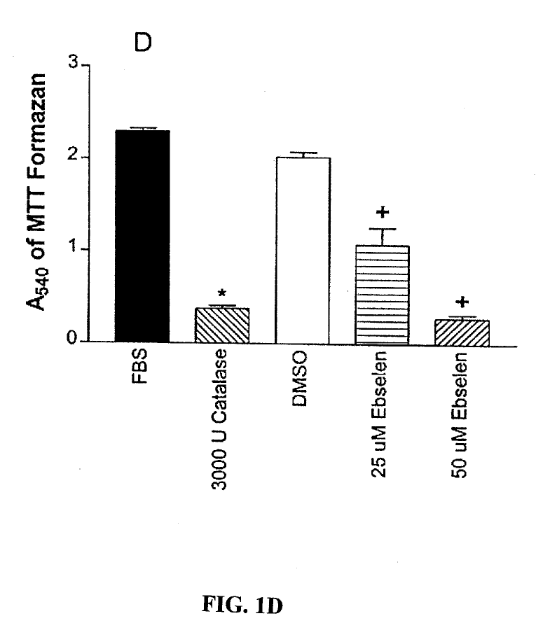

The results of A-C are illustrated in FIG. 1A-1D.

D. 3000 U/ml Catalase, 2 μl/well DMSO, 25 μM Ebselen, and 250 μM dicumarol were tested against Melanoma M1585 cells, Adeno-squamous lung carcinoma NCI-H596 cells, LNCaP FGC prostate adenocarcinoma cells, and breast carcinoma MDA-MB-453 cells for their effects on cell proliferation. Cells stimulated with 10% FBS were plated at a density of 50,000 cells per well. DMSO and antioxidants were added to cell wells at the indicated concentrations. After 48 hours (M1585, H596, and LNCaP) or 72 hours (MDA-MB-453), proliferation was quantitated as described above. The results are summarized in Table I, in which each value represents mean±SE percent inhibition of growth compared to FBS or FBS+DMSO treated control cultures. Percent inhibition of growth was calculated as 100×(1.0−A₅₄₀ of MTT formazan in antioxidant-treated cells/mea A₅₄₀ of MTT formazan of control cells). Each value represents a mean of at least 4 experiments.

TABLE 1
Antioxidant Strategies Reduce Proliferation Of Malignant Cells

| | Percent Growth Inhibition | | |
|---|---|---|---|
| | Antioxidant Strategy | | |
| | Catalase | Ebselen | Dicumarol |
| Cell Line | 3000 U/ml | 25 μM | 250 μM |
| Melanoma M1585 | 80 ± 1* | 94 ± 2* | 99 ± 0* |
| Adeno-squamous lung carcinoma | 69 ± 4* | 57 ± 2* | 97 ± 0* |
| NCI-H596 | | | |
| LNCaP.FGC prostate | 43 ± 7* | 100 ± 0* | 98 ± 1* |
| adenocarcinoma | | | |
| Breast carcinoma MDA-MD-453 | 49 ± 2* | 69 ± 4* | 99 ± 0* |

*p < 0.001 compared to vehicle control cells.

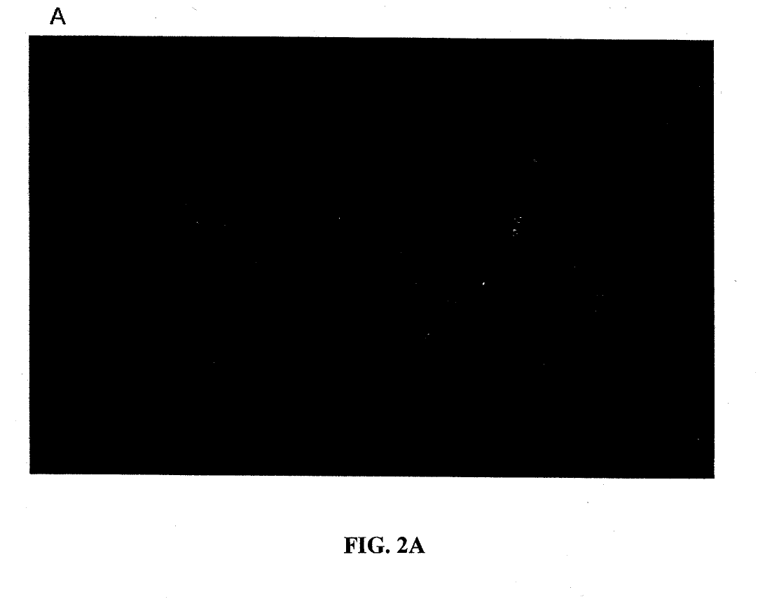

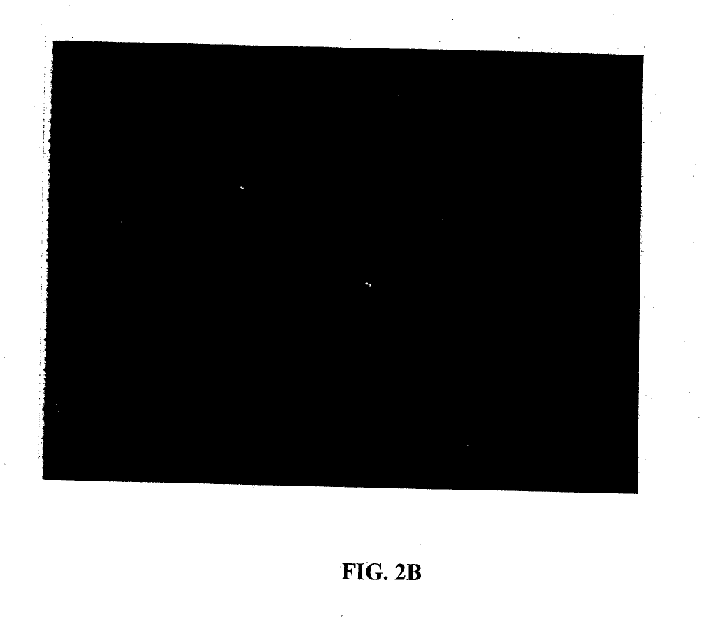

As illustrated in FIG. 1 and Table 1, at concentrations we have previously reported to inhibit growth of cultured human airway smooth muscle (See Brar et al., J. Biol. Chem., 274:20017-20026 (1999)), N-acetylcysteine (NAC) and catalase reduced proliferation of M1619 melanoma cells when added to culture medium (FIG. 1A). In contrast, copper-zinc superoxide dismutase (SOD) had no effect on cell growth. Growth inhibition from catalase was dose-dependent (FIG. 1A), was shared by a variety of catalase preparations from different sources (data not shown) and was eliminated by protein inactivation (FIG. 1B). Catalase treatment did not induce apoptosis (Experiment 2 as illustrated in FIGS. 2A and B), but significantly increased trypan blue dye exclusion. M1619 proliferation was also dramatically reduced by the glutathione peroxidase mime ebselen (FIG. 1C). Catalase and ebselen were antiproliferative even if added 24 hours after melanoma cells were plated (FIG. 1D). Taken together, these results indicate that reactive oxygen species may be important signaling molecules for growth of malignant cell lines and suggest that the proximate growth-signaling form of reactive oxygen may be H₂O₂.

### EXPERMENT 2

Antioxidants do not produce apoptosis or necrosis in malignant human cell lines.

Apoptosis was studied by terminal deoxynucleotidyl transferase (TdT) dependent 3′BOH fluorescein end-labeling of DNA fragments, using a Fluorescein-FragE™ DNA fragmentation detection kit (Oncogene Research Products). As shown in FIG. 2, compared to DNase-treated positive control cells (FIG. 2A), treatment with 3000 U/ml catalase for 24 hours (FIG. 2B) did not induce apoptosis in cultured M1619 cells.

### EXPERIMENT 3

Antioxidant treatment reduces constitutive nuclear DNA binding activity for NF-κB in malignant cell lines.

Confluent cultures of M1619 cells were lysed, nuclear protein was isolated and electrophoresis mobility shift assay (EMSAs) were performed as described, using ³²P-labeled consensus oligonucleotide 5′-AGTTGAGGGGACTTTCCCAGGC-3′ and 3′-TCAACTCCCCTGAAAGGGTCCG-5′, specific for the p50 component of NF-κB.

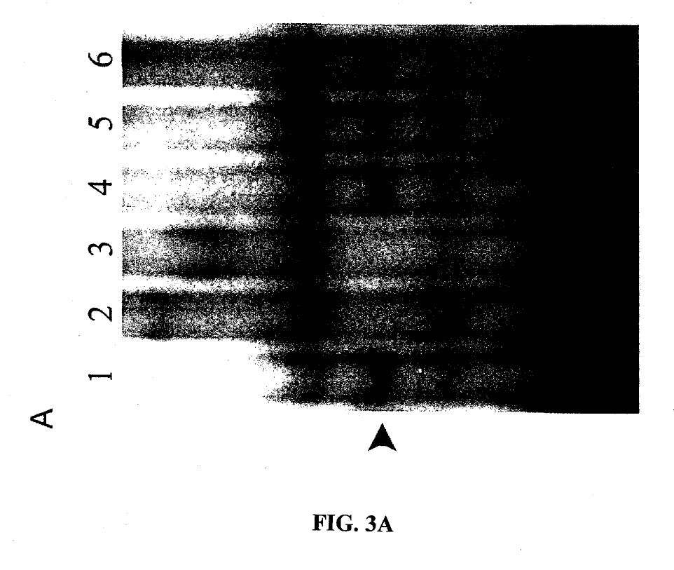

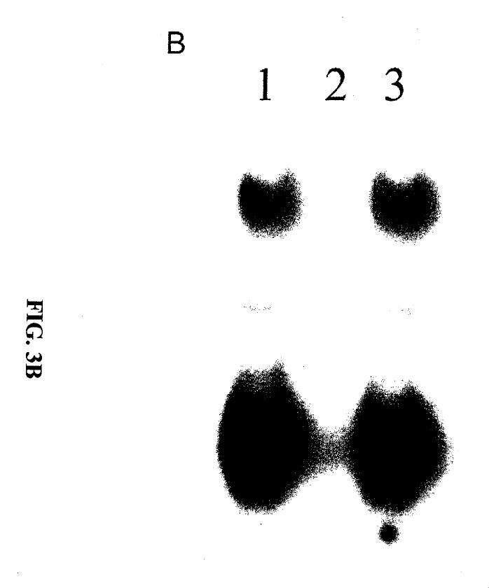

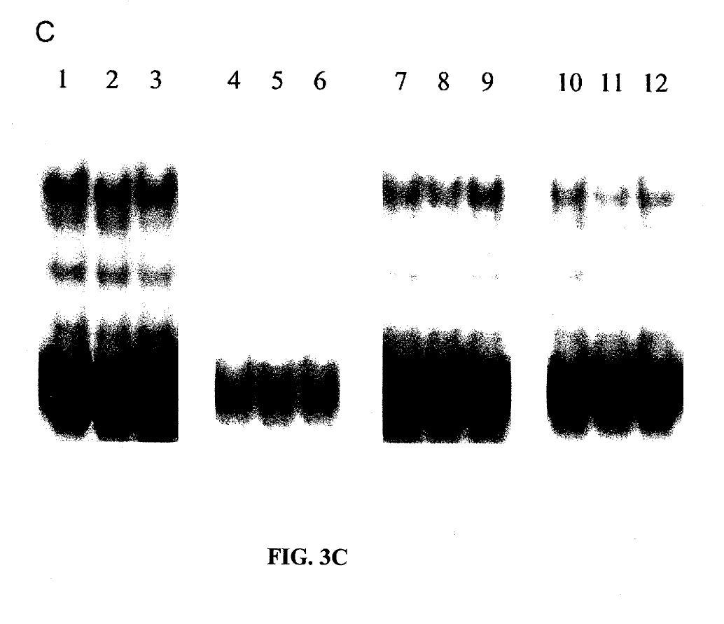

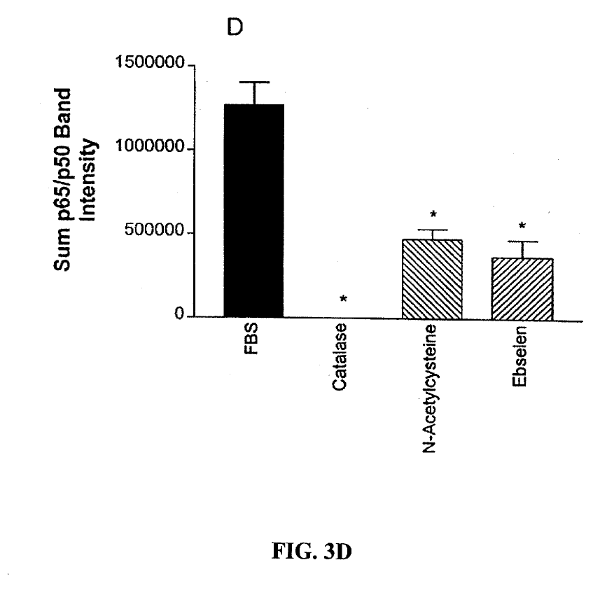

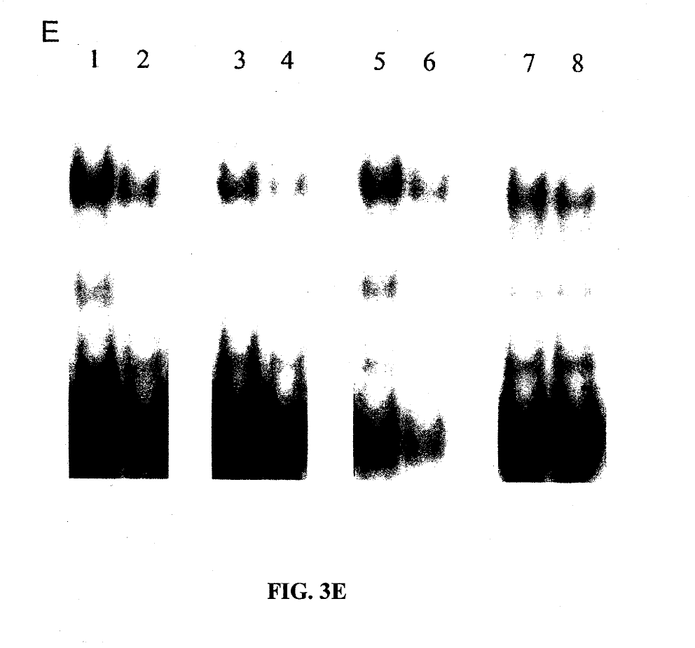

M1619 cells demonstrated prominent constitutive nuclear DNA binding activity for NF-κB (FIG. 3A, lane 1). At least three distinct bands were observed. Supershift experiments demonstrated that the second band (FIG. 3A, lane 1, arrow) contained p65 (lane 2) and p50 (lane 3) NF-κB components, but not p52 (lane 4), Rel-B (lane 5), or c-Rel (lane 6).

For competition assays, M1619 nuclear protein was incubated with ³²P-labeled NF-κB consensus oligonucleotide alone (FIG. 3B, Lane 1), or with ³²P-labeled NF-κB consensus oligonucleotide in addition to 10× unlabeled NF-κB consensus oligonucleotide (FIG. 3B, lane 2), or with ³²P-labeled NF-κB consensus oligonucleotide in addition to 10× unlabeled consensus oligonucleotide specific for cyclic AMP responsive element (CRE) (FIG. 3B, lane 3).

Nuclear DNA binding activity of NF-κB in M1619 cells treated with antioxidants for 24 hours was also assayed. Nuclear protein from untreated positive control cells (FIG. 3C, Lanes 1-3), and from cells treated for 24 hours with 3,000 U/ml catalase (FIG. 3C, lanes 4-6), 20 mM NAC (FIG. 3C, lanes 7-9) or 25 μM Ebselen (FIG. 3C, lanes 10-12) was used in gel shift assay as described above. The p65/p50-containing bands in the gels were quantitatively measured by densitometry and the results are shown in FIG. 3D.

NF-κB Nuclear DNA binding activities in other malignant cell lines including M1585 melanoma cells, LNCaP prostate carcinoma cells, and MDA-MB-453 breast carcinoma cells, treated with catalase (3,000 U/ml for 24 hours) were also assayed with methods described above. The results are shown in FIG. 3E. (Lane 1, untreated M1585 melanoma cells; lane 2, M1585 cells treated with catalase; lane 3, untreated LNCaP prostate carcinoma cells; lane 4, LNCaP cells treated with catalase; lane 5, untreated MDA-MB-453 breast carcinoma cells; lane 6, MDA.MB-453 cells treated with catalase.)

In addition, the effect of serum deprivation on the constitutive nuclear DNA binding activity of NF-κB in M1619 cells was also determined. Near confluent cells were incubated in the presence (FIG. 3E, lane 7) or absence (FIG. 3E, lane 8) of 10% FBS. After 24 hours, nuclear protein was isolated and EMSAs were performed as in described above.

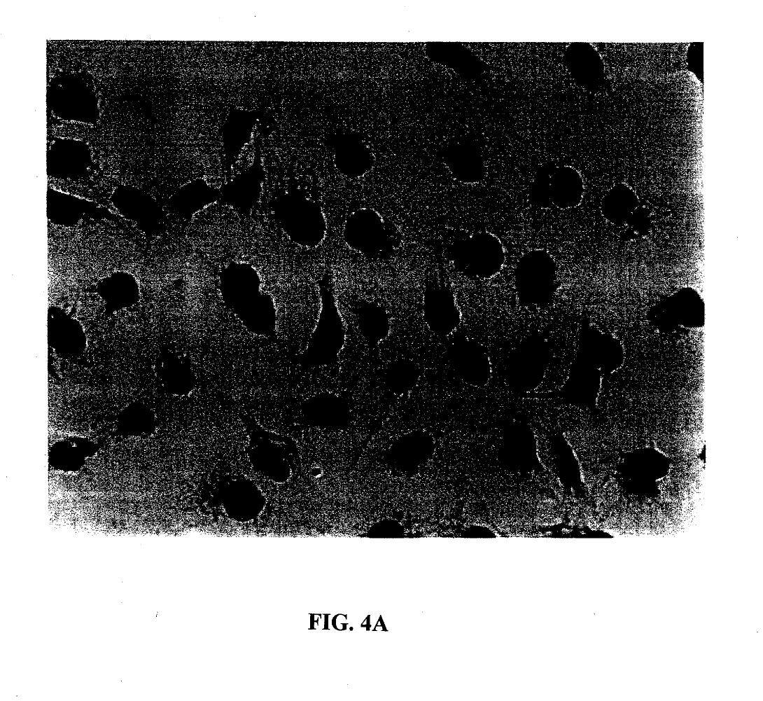

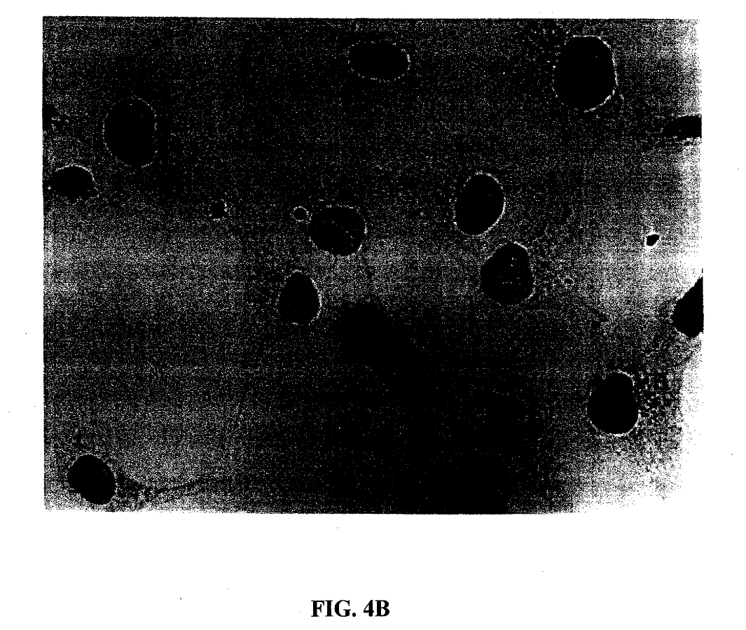

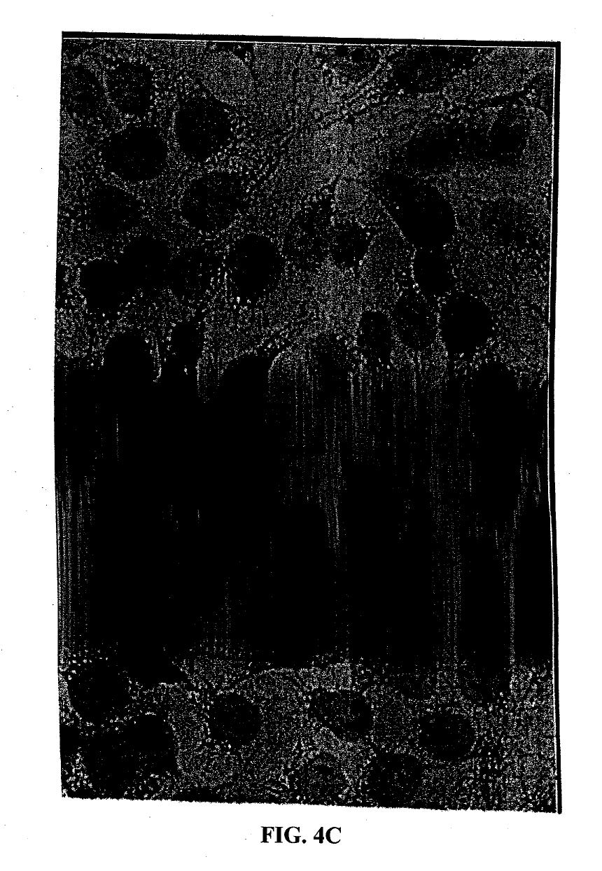

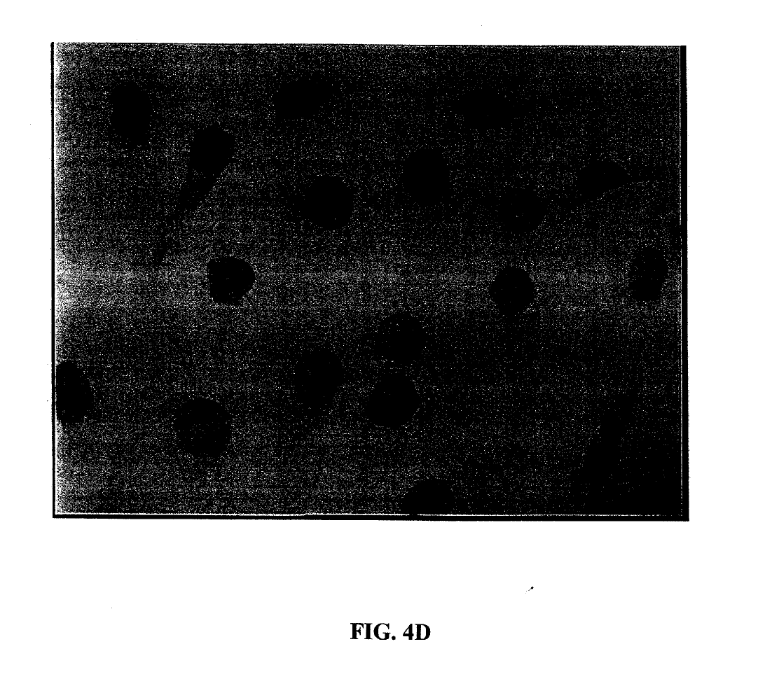

M1619 cells demonstrated prominent constitutive nuclear DNA binding activity for NF-κB nuclear protein (FIGS. 3A and B, lane 1). Several distinct bands were observed, all of which were eliminated by addition of excess specific unlabeled NF-κB consensus oligonucleotides to the binding reaction (FIG. 3B, lane 2). Supershift experiments demonstrated that the second band (FIG. 3A, arrow) contained p65 (FIG. 3A, lane 2) and p50 (FIG. 3A, lane 3) NF-κB components, but not p52, Rel-B or c-Rel (FIG. 3A, lanes 4-6). Constitutive nuclear translocation of NF-κB was confirmed immunohistochemically by intense staining for p65 in M1619 nuclei. See FIG. 4A discussed below. Treatment of cells for 24 hours with the antioxidants catalase, NAC or Ebselen substantially reduced constitutive NF-κB DNA binding activity in nuclear protein (FIGS. 3C and D). Furthermore, exposure of cells to catalase for 24 hours also essentially eliminated immunohistochemical staining for p65 in cell nuclei (FIG. 4B). In addition, catalase treatment for 24 hours also suppressed constitutive nuclear DNA binding activity for NF-κB in M1585 melanoma (FIG. 3E, lane 2), prostate carcinoma (FIG. 3E, lane 4) and breast carcinoma (FIG. 3E, lane 6). These results suggest that constitutive nuclear activation of NF-κB in malignant cell lines may be the consequence of endogenous redox stress.

Serum deprivation slightly decreased, but did not eliminate, constitutive nuclear activation of NF-κB, suggesting that the oxidant stress inducing NF-κB activation is not induced by components of serum, but is endogenous to the malignant cell.

### EXPERIMENT 4

Antioxidants inhibits the nuclear translocation of NF-κB in tumor cells.

Confluent M1619 cells were fixed in paraformaldehyde, permeabilized stained using an antibody to the p65 component of NF-κB and a streptavidin-biotin-immunoperoxidase based method outlined in the text, viewed under light microscopy using a green filter to enhance contrast and photographed at 980× magnification. Control untreated cells showed intense brown staining in nearly all nuclei, corresponding to the presence of anti-p65. See FIG. 4A. In contrast, cells treated for 24 hours with 3,000 U/ml catalase demonstrated anti-p65 brown staining in cytoplasm but little staining in nuclei. See FIG. 4B. The nuclei from catalase treated cells also display greater detail, with prominent nucleoli, not seen in untreated cells shown. In addition, cells treated for 24 hours with 150 μg/ml of apocynin (FIG. 4C) and 250 μM dicumarol (FIG. 4D) were also studied using anti-p65 by the same method. Like cells treated with catalase, these cells demonstrate anti-p65 brown staining in cytoplasm but little staining in nuclei. These cells also display prominent nucleoli.

### EXPERIMENT 5

Catalase decreases the amount of IκBα that is phosphorylated.

M1619 cells either treated with catalase with 3,000 U/ml for 24 hours or untreated were used in immunoassays for phosphorylated IκBα (IκBα-P) using phospho-specific antibody as described above.

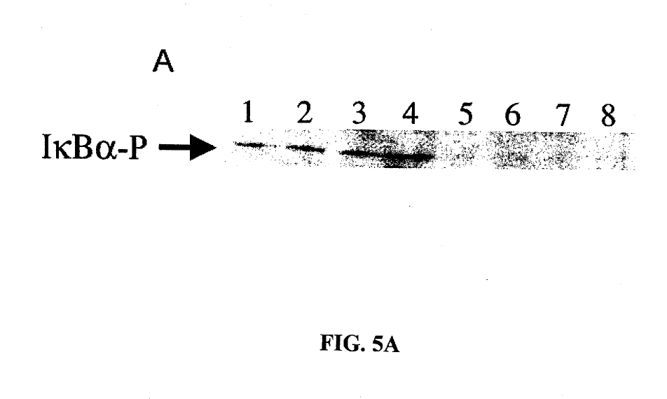

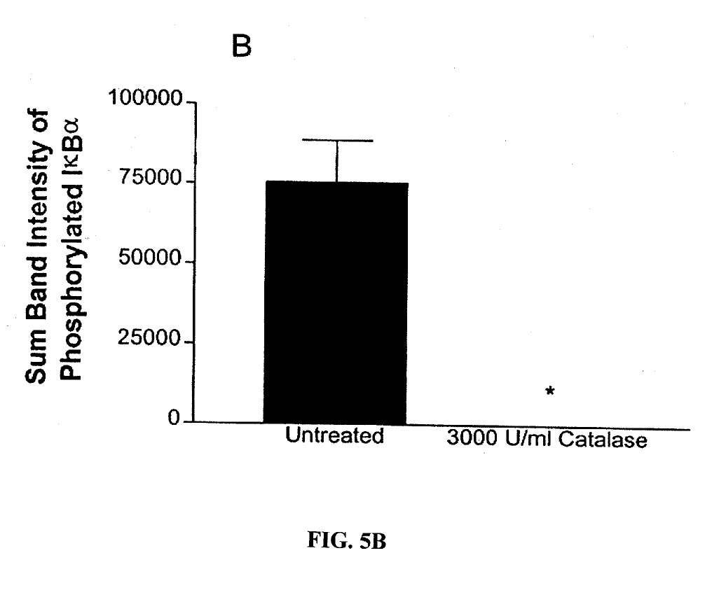

FIG. 5A shows the immunoassays results for phosphorylated IκBα (IκBα-P) in untreated M1619 cells (lanes 1-4) and in M1619 cells treated for 24 hours with 3,000 U/ml catalase (lanes 5-8). Mean ratios of the densitometrically determined sum intensities of IκBα-P staining in the immunoassay experiments are shown in FIG. 5B.

### EXPERIMENT 6

Antioxidants reduce polyploidy, increase S-phase fraction and decrease levels of cyclin B1 and cdc2 kinase in M1619 cells.

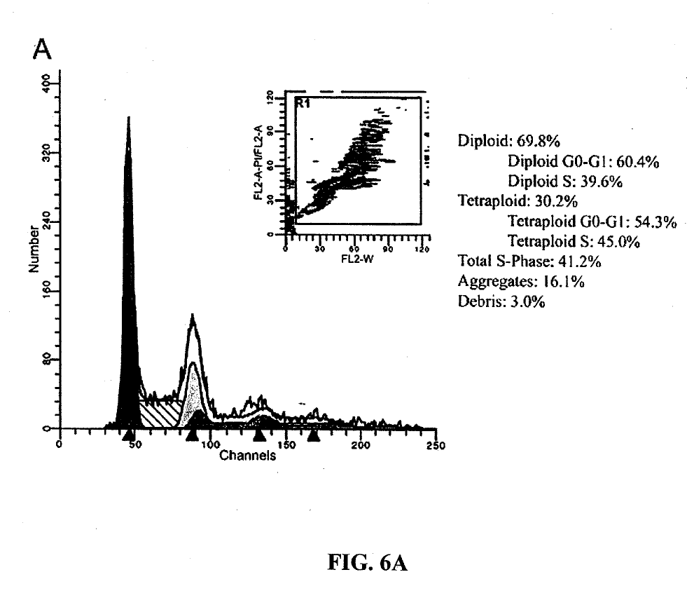

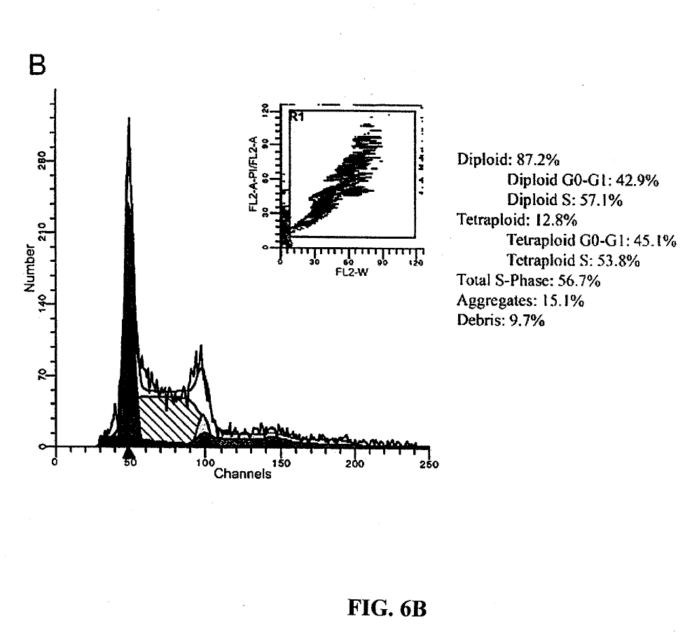

Near confluent monolayers of M1619 cells were incubated in RPMI 1640 and 10% FBS in the presence or absence of 3,000 U/ml catalase. After 24 hours, cells were harvested, ethanol-fixed, permeabilized with proteinase K, stained with propidium iodide and subjected to DNA cell cycle analysis. Whereas a large fraction (30.2%) of untreated cells (FIG. 6A) were tetraploid, only 12.8% of catalase-treated cells (FIG. 6B) were tetraploid. Antioxidant treatment also increased the total percentage of cells in S-phase from 41.2% in untreated controls (FIG. 6A) to 56.7% in cells treated with catalase (FIG. 6B), suggesting a slowing of progression into the G₂-M phase of the cell cycle. G₂-M was hidden by tetraploid G₀-G₁ or by debris and could not be analyzed.

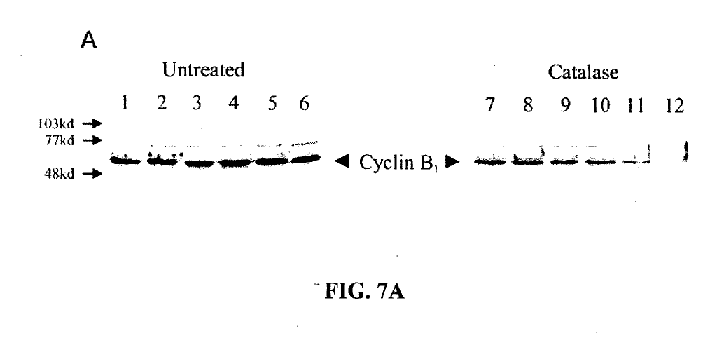

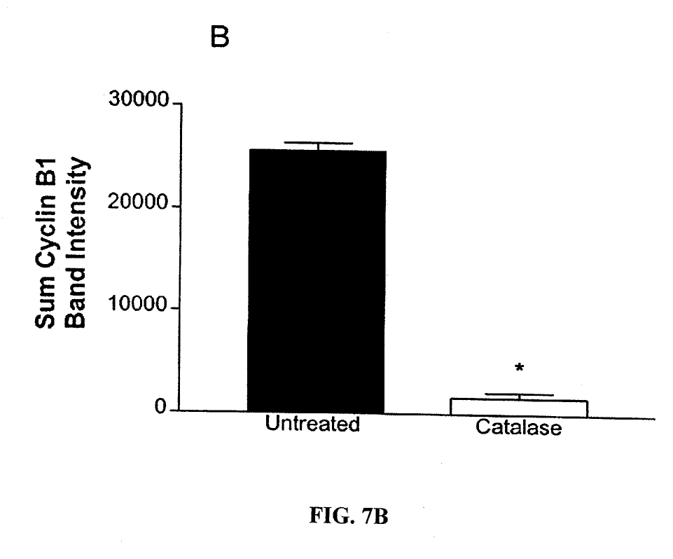

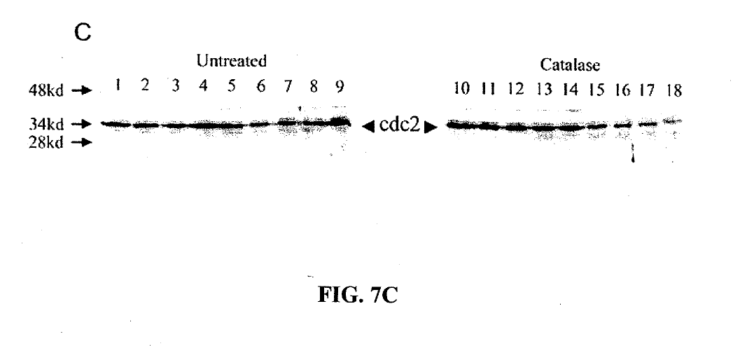

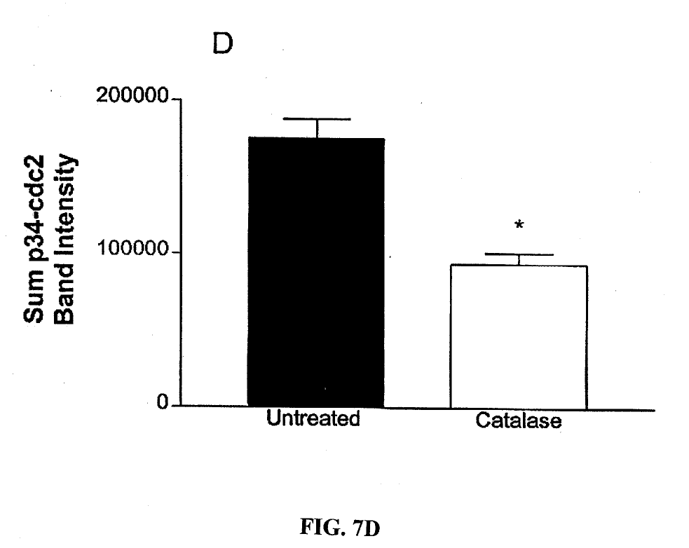

M1619 cells were incubated with or without 3,000 U/ml catalase for various predetermined times (FIG. 7A, lanes 1-6 and 7-12 represent treatment for 2, 4, 8, 12, 24 or 48 hours of untreated control or catalase-treated cells, respectively. FIG. 7C, lanes 1-9 and 10-18 represent treatment for 15 minutes, 30 minutes and 1, 1.5, 2, 4, 8, 12 or 24 hours, of untreated control or catalase-treated cells, respectively.). Immunoblots of cell lysate were performed to quantitate protein levels of cyclin B1 and its associated p34-cdc2 kinase. Densitometry measurements were also taken (FIG. 7B and 7D).

As is clear from FIG. 6A, untreated M1619 melanoma cells are a rapidly proliferating, desynchronized malignant line composed of both diploid and tetraploid cells. Over 30% of cells are tetraploid (FIG. 6A). Treatment with catalase for 24 hours (FIG. 6B) substantially reduces the fraction of tetraploid cells (12.8%) and increases the total fraction of cells in S-phase from 41.2 (untreated) to 56.7% (catalase-treated). Similar changes were seen after treatment with NAC (data not shown). This suggests the possibility that antioxidants impair progression into G₂-M.

To explain these changes in cell cycle kinetics, we compared protein levels of the cell cyclins, cyclin-associated kinases, cyclin kinase inhibitors and the pro-apoptotic regulator p53 in catalase-treated cells to levels in controls. Catalase treatment did not decrease protein levels of the cyclins D1, E and A, or the cyclin-associated kinase cdk2, or increase levels of p53 or the cyclin kinase inhibitors p21^{WAF1/Cip1} or p27 (data not shown). However, catalase produced a significant decrease in expression of cyclin B1 and its associated kinase p34-cdc2 (FIGS. 7A-D). These two proteins, which comprise the regulatory subunit and active kinase of mitosis promoting factor (MPF), accumulate during interphase and peak at the G₂-M transition, are critical for the proper timing of a cell=s entry into mitosis. Therefore, their reduction provides a potential basis for the reduced fraction of tetraploid cells and increased fraction of S-phase cells following treatment of melanoma cells with antioxidants.

### EXPERIMENT 7

Ferricytochrome c reduction and cellular proliferation of melanoma cells are reduced by quinone analogs and NAD(P)H oxidase inhibitors.

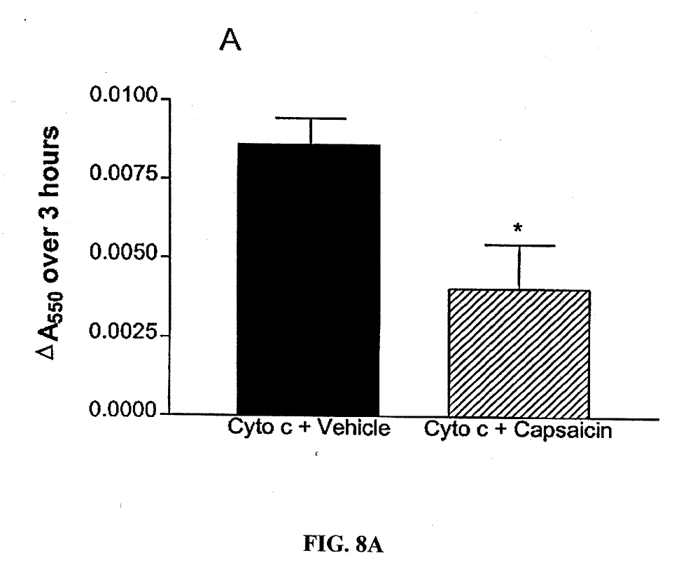

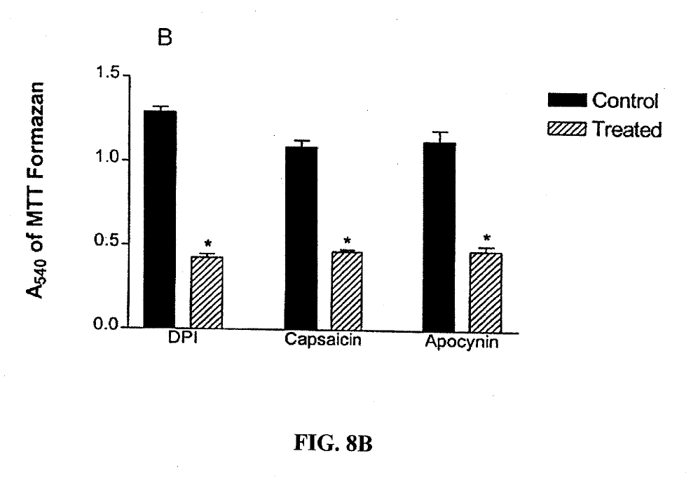

A. Capsaicin inhibits ferricytochrome c reduction. M1619 cells grown on 24-well plates were washed with DPBS, and incubated in 5% CO₂/air at 37° C. with 160 μM ferricytochrome c in total volume of 550 μl of HBSS with or without the quinone analog capsaicin (100 μM final concentration) added in 5 μl/ml of ethanol. The absorbance of each well was measured at 550 nm initially and 3 hours later using an ELx800 UV automated microplate reader (Biotek Instruments, Highland Park, Vt.). The results are illustrated in FIG. 8A.

B. Capsaicin and NAD(P)H oxidase inhibitors decrease melanoma cell proliferation. Cells stimulated with 10% FBS were plated at a density of 50,000 cells per well and inhibitors were added to wells in the following final concentrations and vehicles: diphenylene iondonium chloride, 25 μM in DPBS, capsaicin, 100 μM in 5 μl/ml of ethanol; and apocynin.150 μl/ml in 5 μl/ml of DMSO. After 48 hours, proliferation was quantitated as described above. The results are illustrated in FIG. 8B.

M1619 cells had no measurable xanthine oxidase activity, and no evidence was detected of mRNA specific for the p22 and gp91^{phox} components of neutrophil NADPH oxidase or for the newly described mox-1 (or NOH-1L) oxidase. Neither cellular reduction of ferricytochrome c nor proliferation were reduced by the xanthine oxidase inhibitor allopurinol, the cycloxygenase inhibitor indomethacin, the cytochrome P450 inhibitor cimetidine, the nitric oxide synthase inhibitor_{ω}-nitro-L-arginine or the mitochrondrial respiratory chain inhibitor retenone. However, ferricytochrome c reduction was significantly decreased by the quinone analog capsaicin (FIG. 8A). Capsaicin, as well as the NAD(P)H oxidase inhibitors diphenylene iodonium chloride (DPI) and apocynin (4′-hydroxy-3′-methoxy-acetophenone), significantly reduced proliferation of M1619 melanoma cells at 48 hours (FIG. 8B). Also, apocynin treatment of cells for 24 hours reduced constitutive nuclear translation of NF-κB as assessed by immunohistochemistry (FIG. 4C vs FIG. 4A). Taken together these results suggested that the source of endogenous O₂ generation stimulating NF-κB activation and influencing cellular proliferation in this cell line was an NAD(P)H oxidoreductase activity distinct from the gp91^{phox} neutrophil NADPH oxidase, mox-1 or the NOH-1L oxidase.

### EXPERIMENT 8

Ferricytochrome c reduction, NF-κB activation, and cellular proliferation of melanoma cells are reduced by dicumarol.

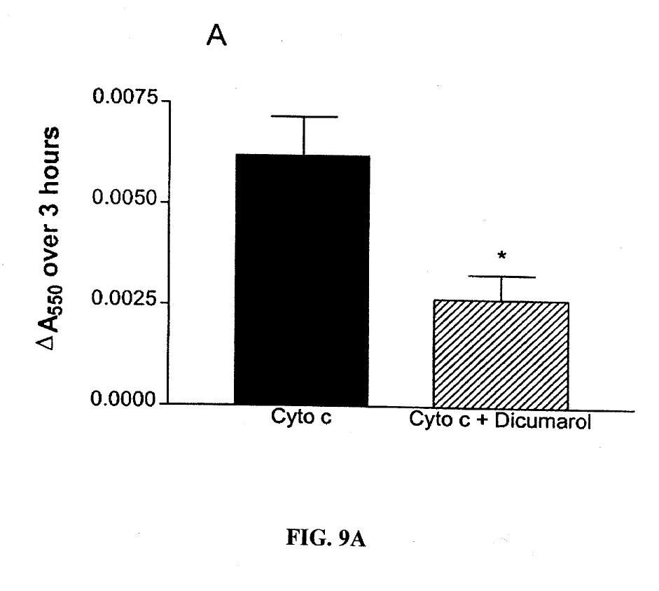

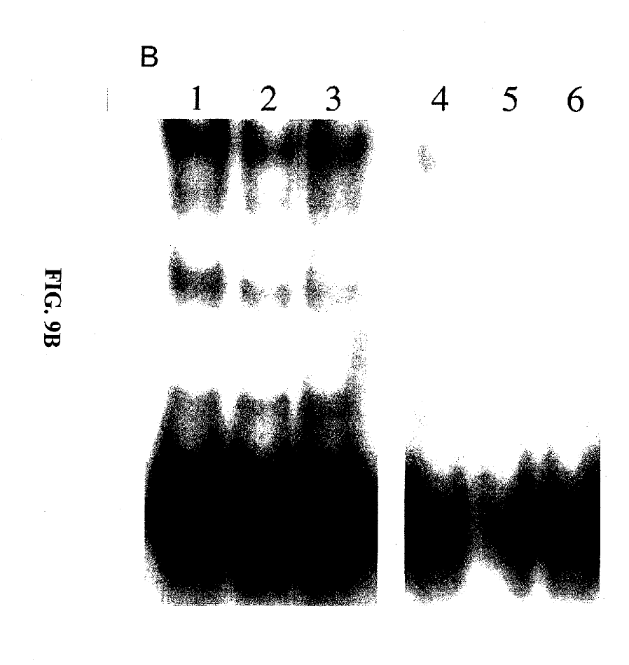

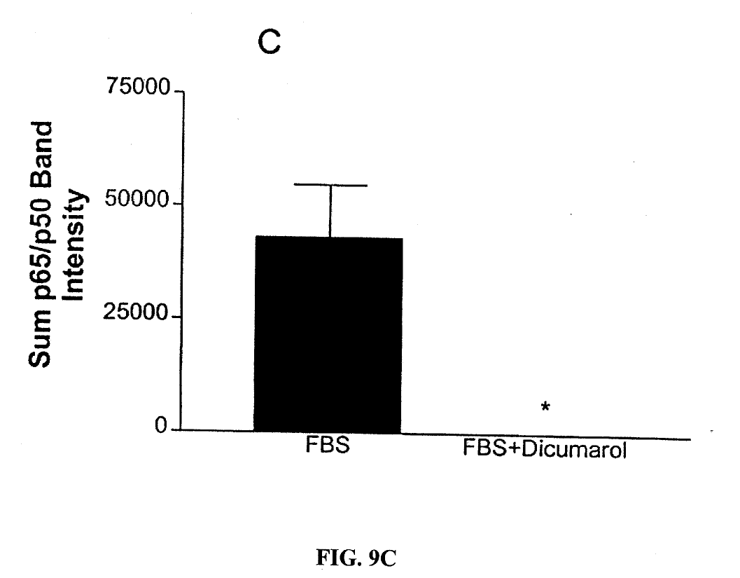

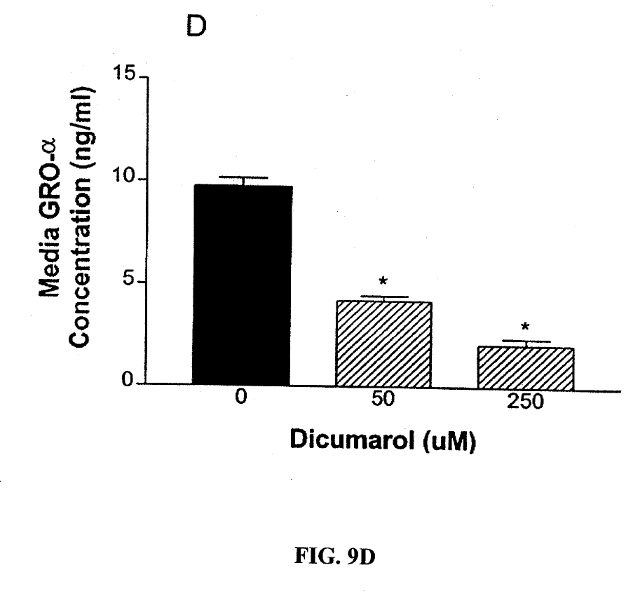

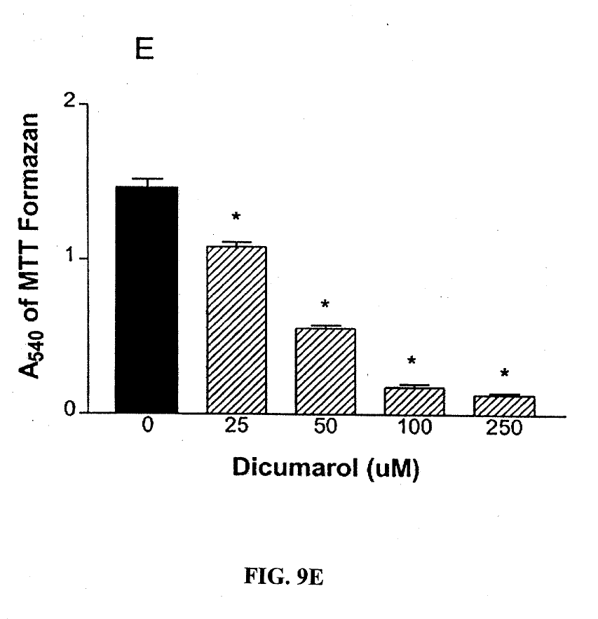

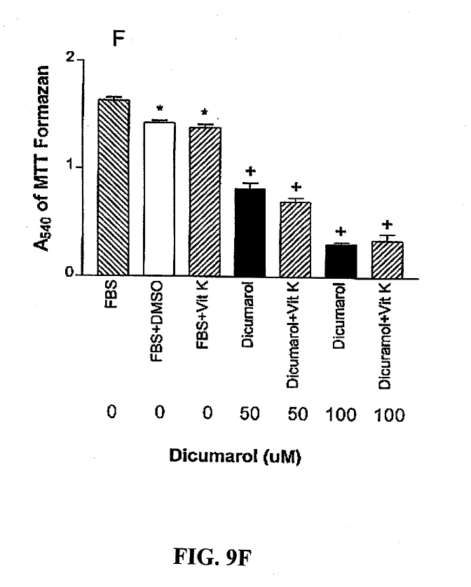

A. The specific NAD(P)H:quinone oxidoreductase inhibitor dicumarol inhibits ferricytochrome c reduction by M1619 cells. Dicumarol (250 μM) was added to confluent M1619 cell cultures in complete medium before each experiment. After 60 min, dicumarol-containing medium was removed, cells were washed with DPBS, and ferricytochrome c reduction was studied as described in Experiment 7A. A 50 mM concentration of dicumarol was dissolved in water by drop-wise addition of 0.1 N NaOH. Addition of up to 2.5 μl of this solution per ml (250 μM final concentration) did not change the pH of complete medium. The results are illustrated in FIG. 9A.

B. Dicumarol reduces constitutive NF-κB activation in M1619 cells. Near confluent cultures of M1619 cells incubated with complete medium alone or medium containing 250 μM dicumarol for 24 hours. Cells were then lysed, nuclear protein was isolated and electrophorectic mobility shift assays (EMSAs) were performed as described above. The radiograph is shown in FIG. 9B. Constitutive NF-κB DNA binding was greatly reduced in dicumarol treated cells (lanes 4-6) compared to cells incubated in growth medium alone (lanes 1-3). Densitometry measurements of the p65/p50-containing bands from the gels in FIG. 8B were taken and are summarized in FIG. 9C.

C. Dicumarol inhibits melanoma cell production of the autocrine growth factor GRO-α. Near confluent M1619 cells were incubated with or without dicumarol at the concentrations indicated. After 24 hours, GRO-α concentration was measured in media. The results are shown in FIG. 9D.

D. Dicumarol inhibits proliferation of M1619 cells. Cells stimulated with 10% FBS were plated at a density of 50,000 cells per well and dicumarol was added to medium in the concentrations shown. After 48 hours, proliferation was quantitated as described and the results are illustrated in FIG. 9E.

E. Vitamin K does not prevent growth inhibition from dicumarol. M1619 cells stimulated with 10% FBS were plated at a density of 50,000 cells per well and dicumarol or dicumarol plus an equimolar concentration of vitamin K₂ were added to medium in the concentrations shown. The vehicle for vitamin K₂ (5 μl per ml of DMSO) was added to all wells. After 48 hours, proliferation was quantitated as described and the results are illustrated in FIG. 9F. Dicumarol alone versus dicumarol+vitamin K₂ were not different at either concentration.

One infrequently-considered NAD(P)H dependent source of reactive oxygen species is NAD(P)H:(quinone acceptor)oxidoreductase (EC 1.6.99.2), a homodimeric ubiquitous cytosolic and membrane flavoprotein that catalyzes the two electron reduction of quinones, including membrane ubiquinone (See Ernster, Methods Enzymol. 10:309-317 (1967)), which can, in turn, redox cycle with molecular oxygen to produce O₂. Like other flavoenzymes, it is inhibited by diphenylene iodonium. See O'Donnell et al., Mol. Pharmacol 46:778-786 (1994). It differs from other quinone reductases in the cell in that it uses both NADH and NADPH as cofactors and is selectively inhibited by low concentration of dicumarol, a compound previously used as an anticoagulant to disrupt production of vitamin-K dependent clotting factors. See Edwards et al. Biochem. J. 187:429-436 (1980). Dicumarol significantly decreased ferricytochrome c reduction by cultured M1619 cells (FIG. 9A). Dicumarol also substantially reduced constitutive activation of NF-κB in melanoma cells, studied by both electrophoretic mobility shift assay (FIGS. 9B and C) or immunohistochemistry (FIG. 2D). In addition, dicumarol inhibited the functionality of NF-κB in melanomas, shown by the dramatic reduction of GRO-α protein expression in dicumarol-treated cells (FIG. 9D). Dicumarol treatment reduced tumor cell proliferation in a dose-dependent fashion (FIG. 9E and Table 1). Tumor cell growth inhibition by dicumarol was not from interference with a previously unrecognized aspect of vitamin K metabolism, since addition of equimolar concentrations of vitamin K to growth medium did not impair the growth inhibiting effect of dicumarol (FIG. 8F). The growth inhibitory effect of dicumarol may also relatively specific for tumor cells, since it failed to significantly reduce proliferation of normal human airway myocytes (only 8±4% inhibition of growth at 48 hours with 250 μM), another cell line for which was have previously found reactive oxygen species important as growth-signaling intermediates. Brar et al. J. Biol. Chem., 274:20017-20026 (1999). This suggests that the redox couple between NAD(P)H:quinone oxidoreductase and ubiquinone may be relatively more important as a source of growth-signaling reactive oxygen species in transformed neoplastic cells than in normally regulated nonmalignant tissues. a specific inhibitor of NAD(P)H:quinone oxidoreductase (DT-diaphorase).

Although the foregoing invention has been described in some detail by way of illustration and example for purposes of clarity of understanding, it will be obvious that certain changes and modifications may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed is:

1. A method for treating cancer in a patient, comprising administering to the patient a therapeutically effective amount of dicumarol or analog thereof and a vitamin K or analog thereof.

2. The method of claim 1, wherein an analog of dicumarol is administered, the analog being selected from the group consisting of phenprocoumon (4-hydroxy-3-(1-phenylpropyl)-2H-1-benzopyran-2-one), warfarin (3-(α-acetonylbenzyl)-4-hydroxycoumarin), 7-[Diethylamino]-4-trifluoromethylcoumarin, 7-amino-4-trifluoromethylcoumarin, 7-amino-4-methylcoumarin, 4-methylcoumarin, 7-hydroxy-4-methylcoumarin, 7,8-dihydroxy-4-methylcoumarin, and 1,1-dimethyl-alloylcoumarin, and derivatives and analogs thereof.

3. The method of claim 1, wherein the vitamin K or analog thereof is selected from the group consisting of phylloquinone (vitamin K₁), menaquinone (vitamin K₂), menadione (vitamin K₃), vitamin K₅, dihydrovitamin K, menaquinone-4, and derivatives and analogs thereof

4. The method of claim 1, wherein the cancer is selected from the group consisting of carcinomas, myelomas, melanomas, and lymphomas.

5. The method of claim 1, wherein the cancer is selected from the group consisting of melanoma, non-small cell lung cancer, small cell lung cancer, renal cancer, colorectal cancer, breast cancer, pancreatic cancer, gastric cancer, bladder cancer, ovarian cancer, uterine cancer, lymphoma, and prostate cancer.

6. The method of claim 1, wherein the cancer is selected form the group consisting of melanoma, lung cancer, breast cancer, and prostate carcinoma.

7. The method of claim 1, wherein the dicumarol or analog thereof and the vitamin K or analog thereof are administered simultaneously in the same or separate pharmaceutical preparations.

8. The method of claim 1, wherein the dicumarol or analog thereof and the vitamin K or analog thereof are administered in equimolar concentration.

9. The method of claim 1, wherein the dicumarol or analog thereof and the vitamin K or analog thereof are administered in a pharmaceutically acceptable carrier.

10. The method of claim 1, wherein the dicumarol or analog thereof and the vitamin K or analog thereof are administered via a route selected from the group consisting of parenteral, intravenous, oral, intradermal, subcutaneous, and topical administration.

11. The method of claim 1, wherein the dicumarol or analog thereof is administered in an amount of about 0.1 mg to about 5000 mg per day.

12. The method of claim 1, wherein the dicumarol or analog thereof is administered in an amount of about 10 mg to about 1000 mg per day.

13. The method of claim 1, wherein the vitamin K or analog thereof is administered in an amount of about 0.1 mg to about 500 mg per day.

14. The method of claim 1, wherein the vitamin K or analog thereof is administered in an amount of about 0.5 mg to about 200 mg per day.

15. The method of claim 1, further comprising administering to the patient another anticancer agent.

16. The method of claim 15, wherein the anticancer agent is selected from group consisting of cisplatin, carboplatin, cyclophosphamide, nitrosoureas, fotemustine, herceptin, carmustine, vindesine, etoposide, daunorubicin, adriamycin, taxol, taxotere, fluorouracil, methotrexate, melphalan, bleomycin, salicylates, aspirin, piroxicam, ibuprofen, indomethacin, maprosyn, diclofenac, tolmetin, ketoprofen, nabumetone, oxaprozin, doxorubicin, nonselective cyclooxygenase inhibitors, and selective cyclooxygenase-2 (COX-2) inhibitors.

17. A method for treating a cancer selected from the group consisting of carcinomas, myelomas, melanomas, and lymphomas in a patient, comprising administering to the patient dicumarol in an amount effective to suppress NAD(P)H:quinnone oxidoreductase activity and inhibit constitutive activation of NF-κB in cancer cells, and administering a vitamin K in an amount effective to offset the anticoagulant effect of dicumarol.

18. The method of claim 17, further comprising administering to the patient another anticancer agent.

19. The method of claim 18, wherein the anticancer agent is selected from the group consisting of cisplatin, cyclophosphamide, etoposide, duanorubicin, adriamycin, fluorouracil, and carboplatin.

20. The method of claim 17, wherein the dicumarol is administered in an amount of about 10 mg to about 1000 mg per day and the vitamin K is administered in an amount of about 0.5 mg to about 200 mg per day.
